SEQUENCE LISTING (7 sequences)

SEQ ID NO: 1 - DNA, 306 bp, homo sapiens
  CDS (1)..(306)
atgaagctcttatctttggtggctgtggtcgggtgtttgctggtgcccccagctgaagccaacaagagttctgaagatatccggtgcaaatgcatctgtccaccttatagaaacatcagtgggcacatttacaaccagaatgtatcccagaaggactgcaactgcctgcacgtggtggagcccatgccagtgcctggccatgacgtggaggcctactgcctgctgtgcgagtgcaggtacgaggagcgcagcaccaccaccatcaagtgggcctcaccctccagggaccagatgcctgagacctga

SEQ ID NO: 2 - protein, 101 aa, homo sapiens
MKLLSLVAVVGCLLVPPAEANKSSEDIRCKCICPPYRNISGHIYNQNVSQKDCNCLHVVEPMPVPGHDVEAYCLLCECRYEERSTTTIKWASPSRDQMPET

SEQ ID NO: 3 - DNA, 246 bp, homo sapiens
aacaagagttctgaagatatccggtgcaaatgcatctgtccaccttatagaaacatcagtgggcacatttacaaccagaatgtatcccagaaggactgcaactgcctgcacgtggtggagcccatgccagtgcctggccatgacgtggaggcctactgcctgctgtgcgagtgcaggtacgaggagcgcagcaccaccaccatcaagtgggcctcaccctccagggaccagatgcctgagacctga

SEQ ID NO: 4 - protein, 80 aa, homo sapiens
NKSSEDIRCKCICPPYRNISGHIYNQNVSQKDCNCLHVVEPMPVPGHDVEAYCLLCECRYEERSTTTIKWASPSRDQMPE

SEQ ID NO: 5 - protein, 87 aa, homo sapiens
NKSSEDIRCKCICPPYRNISGHIYNQNVSQKDCNCLHVVEPMPVPGHDVEAYCLLCECRYEERSTTTIKWASPSRDQMPETHHHHHH

SEQ ID NO: 6 - DNA, 252 bp, homo sapiens
gaagccaacaagagttctgaagatatccggtgcaaatgcatctgtccaccttatagaaacatcagtgggcacatttacaaccagaatgtatcccagaaggactgcaactgcctgcacgtggtggagcccatgccagtgcctggccatgacgtggaggcctactgcctgctgtgcgagtgcaggtacgaggagcgcagcaccaccaccatcaagtgggcctcaccctccagggaccagatgcctgagacctga

SEQ ID NO: 7 - protein, 83 aa, homo sapiens
EANKSSEDIRCKCICPPYRNISGHIYNQNVSQKDCNCLHVVEPMPVPGHDVEAYCLLCECRYEERSTTTIKWASPSRDQMPET

(19) United States
(12) Patent Application Publication
(10) Pub. No.: US 2007/0067858 A1
(43) Pub. Date: Mar. 22, 2007

(54) Novel il-8-like polypeptides

(76) Inventors: Gregg Mcallister, Charlestown, MA (US); Regina Gorski, Merchantville, NJ (US); Jadwiga Bienkowska, Cambridge, MA (US)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/557,234

(22) PCT Filed: May 27, 2004
(86) PCT No.: PCT/EP04/05739

§ 371 Date: Oct. 24, 2006

Related U.S. Application Data
(60) Provisional application No. 60/473,634, filed on May 27, 2003.

(51) Int. Cl. A01K 67/027; C12Q 1/68; C07H 21/04; C12P 21/04; C07K 14/54

(52) U.S. Cl. 800/14; 424/85.2; 435/6; 435/69.52; 435/354; 435/320.1; 530/351; 536/23.5

(57) ABSTRACT

The present invention discloses open reading frames (ORFs) in human genome encoding for novel IL-8-like/TMEM9 polypeptides, and reagents related thereto including variants, mutants and fragments of said polypeptides, as well as ligands and antagonists directed against them. The invention provides methods for identifying and making these molecules, for preparing pharmaceutical compositions containing them, and for using them in the diagnosis, prevention and treatment of diseases.

### FIELD OF THE INVENTION

The present invention relates to nucleic acid sequences identified in the human genome as encoding for novel polypeptides, more specifically for IL-8-like/TMEM9 polypeptides.

All publications, patents and patent applications cited herein are incorporated in full by reference.

### BACKGROUND OF THE INVENTION

Many novel polypeptides have been already identified by applying strict homology criteria to known polypeptides of the same family. However, since the actual content in polypeptide-encoding sequences in the human genome for IL-8-like polypeptides (and for any other protein family) is still unknown, the possibility still exists to identify DNA sequence encoding polypeptide having IL-8-like polypeptide activities by applying alternative and less strict homology/structural criteria to the totality of Open Reading Frames (ORFs, that is, genomic sequences containing consecutive triplets of nucleotides coding for amino acids, not interrupted by a termination codon and potentially translatable into a polypeptide) present in the human genome.

The mammalian immune response is based on a series of complex, network-like interactions involving cellular components (such as lymphocytes or granulocytes) and soluble proteins, capable of modulating cellular activities (movement, proliferation, differentiation, etc.). Thus, there is considerable interest in the isolation and characterization of cell modulating factors, with the purpose of providing significant advancements in the diagnosis, prevention, and therapy of human disorders, in particular the ones associated to the immune system.

Chemokines are amongst these soluble proteins, since they are involved in the directional migration and activation of cells. This superfamily of small (70-130 amino acids), secreted, heparin-binding, pro-inflammatory proteins is known especially for the role in the extravasation of leukocytes from the blood to tissue localizations needing the recruitment of these cells (Fernandez E J and Lolis E, Annu. Rev. Pharmacol. Toxicol., 42:469499, 2002).

Chemokines are not only functionally related but also structurally related, since they all contain a central region in which conserved cysteine residues form intramolecular bonds. In particular, the number and the position of the conserved cysteine residues in the N-terminal sequence of the mature polypeptides is the basic criteria for the generally recognized classification of chemokines, essentially divided between chemokines containing isolated or adjacent cysteine residues, or cysteine residues separated by 1-3 amino acids.

A series of membrane receptors, all heptahelical G-protein coupled receptors, are the binding partners that allow chemokines to exert their biological activity on the target cells. The physiological effects of chemokines result from a complex and integrated system of concurrent interactions. Different cells can present specific combinations of receptors according to their state and/or type. Moreover, chemokine receptors often have overlapping ligand specificity, so that a single receptor can bind different chemokines, as well a single chemokine can bind different receptors, still at high affinity.

Usually chemokines are produced at the site of an injury, inflammation, or other tissue alteration, and exert their activity in a paracrine or autocrine fashion. However, cell-type specific migration and activation in inflammatory and immune processes is not the sole activity of chemokines. Other physiological activities, such as hematopoiesis or angiogenesis, and pathological conditions, such as metastasis, transplant rejection, Alzehimer's disease or atherosclerosis, appear to be regulated by at least some of these proteins, since chemokines are found considerably up-regulated and/or activated in several animal models or clinical samples (Haskell C A et al., Curr. Opin. Invest. Drugs, 3: 399-455, 2002; Lucas A D and Greaves D R, Exp. Rev. Mol. Med. 2001; Frederick M J and Clayman G L, Exp. Rev. Mol. Med. 2001; Godessart N and Kunkel S L, Curr. Opin. Immunol., 13: 670-675, 2001; Reape T J and Groot P H, Atherosclerosis, 147:213-25, 1999).

There are potential drawbacks in using chemokines as therapeutic agents (tendency to aggregate and promiscuous binding, in particular), but molecules having antagonistic properties against chemokines are widely considered as offering valuable opportunities for therapeutic intervention in disorders associated to excessive chemokine activities. The inhibition of specific chemokines and their receptors is considered a solution for preventing undesirable or uncontrolled cellular processes, such as recruitment or activation (Baggiolini M, J. Intern. Med., 250: 91-104, 2001; Proudfoot A, et al. Immunol. Rev., 177:246-256, 2000; Rossi D and Zlotnik A, Annu. Rev. Immunol., 18:217-42,2000).

The technologies and information on human genome and physiology now available were also used for discovering novel chemokines and receptors possibly providing new and useful therapeutic molecules and targets. Initially, chemokines genes were regularly mapped on chromosomes 4 and 17, in gene-rich areas of human genome (Nomiyama H et al., Genes Immun, 2: 110-113, 2001), but the literature provides many examples of different approaches for characterizing novel chemokines by making use of bioinformatics analysis of transcripts, which are expressed in lymphoid and other tissues with individually characteristic patterns and mapped to chromosomal loci different from the traditional chemokine gene clusters (WO 02/70706; Wells T N. and Peitsch M C. Methods Mol. Biol., 138: 65-73, 2000; Chantry D F et al., J Leukoc Biol, 64: 49-54, 1998; RossiD et al., J. Immunol, 158:1033-1036, 1997).

Interleukin-8 (IL-8), also called neutrophil-activating peptide-1 or SCYB8, is a tissue-derived peptide secreted by several types of cells in response to inflammatory stimuli.

Interleukin-8 is one of a family of 13 human CXC chemokines. These small basic heparin-binding proteins are proinflammatory and primarily mediate the activation and migration of neutrophils into tissue from peripheral blood. IL-8 has been implicated in the pathogenesis of the viral lower respiratory tract infection bronchiolitis, caused by the respiratory syncytial virus (RSV). This disease is responsible for major epidemics each year, with many thousands of infants requiring hospital treatment. High levels of IL-8 are found in nasal secretions and tracheal aspirates of infants with RSV bronchiolitis, and the level of IL-8 appears to be correlated with disease severity (Smyth, R. L. et al., Arch. Dis. Child. 82 (Suppl. 1): A4-A5, 2000).

IL-8 induces rapid mobilization of hematopoietic progenitor cells (HPCs). Mobilization can be prevented completely in mice by pretreatment with neutralizing antibodies against the beta-2-integrin Lfa1 (CD11A). In addition, murine HPCs do not express Lfa1, indicating that mobilization requires a population of accessory cells.

Pruijt, J. F. M et al. (Proc. Nat. Acad. Sci. 99: 6228-6233, 2002) showed that polymorphonuclear cells (PMNs) serve as key regulators in IL-8-induced HPC mobilization. The role of PMNs was studied in mice rendered neutropenic by administration of a single dose of antineutrophil antibodies. Absolute neutropenia was observed up to 3 to 5 days, with a rebound neutrophilia at day 7. The IL-8-induced mobilizing capacity was reduced significantly during the neutropenic phase, reappeared with recurrence of the PMNs, and was increased proportionately during the neutrophilic phase. The data demonstrated that IL-8-induced mobilization of HPCs requires the in vivo activation of circulating PMNs.

Many novel chemokines have been already identified by applying strict homology criteria to known chemokines. However, since the actual content in polypeptide-encoding sequence in the human genome for chemokines (and for any other protein family) is still unknown, the possibility still exists to identify DNA sequence encoding polypeptide having chemotactic activities by applying alternative and less strict homology/structural criteria to the totality of Open Reading Frames (ORFs, that is, genomic sequences containing consecutive triplets of nucleotides coding for amino acids, not interrupted by a termination codon and potentially translatable in a polypeptide) present in human genome.

The roles that IL-8-like proteins have in diseases are currently under investigation. However, it is clear that the identification of novel IL-8-like proteins is of significant importance in increasing understanding of the underlying pathways that lead to certain disease states in which these proteins are implicated, and in developing more effective gene or drug therapies to treat these disorders.

### SUMMARY OF THE INVENTION

The invention is based upon the identification of an Open Reading Frame (ORF) in the human genome encoding a novel IL-8-like/TMEM9 polypeptide. This polypeptide will be referred to herein as the SCS0010 polypeptide.

Accordingly, the invention provides isolated SCS0010 polypeptides having the amino acid sequence given by SEQ ID NO: 2 and their mature forms, histidine tag forms, variants, and fragments, as polypeptides having the activity of IL-8-like/TMEM9 polypeptides. The invention includes also the nucleic acids encoding them, vectors containing such nucleic acids, and cells containing these vectors or nucleic acids, as well as other related reagents such as fusion proteins, ligands, and antagonists.

The invention provides methods for identifying and making these molecules, for preparing pharmaceutical compositions containing them, and for using them in the diagnosis, prevention and treatment of diseases.

### BRIEF DESCRIPTION OF THE FIGURES

FIG. 1: Alignment of SCS0010 with members of the IL-8-like family.

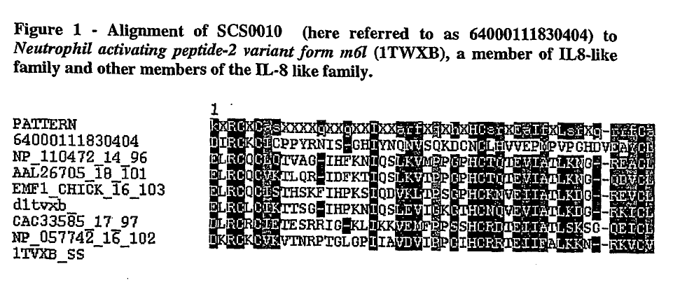

FIG. 2: Intron/Exon structure of SCS0010.

FIG. 3: Nucleotide sequence of SCS0010 with translation.

FIG. 4: Map of pCR4-TOPO-SCS0010.

FIG. 5: Map of pDONR 221.

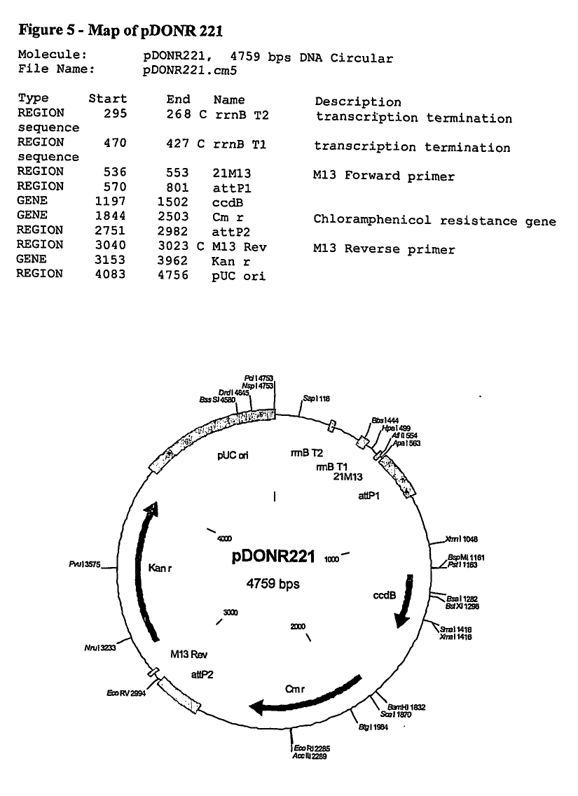

FIG. 6: Map of expression vector pEAK12d.

FIG. 7: Map of Expression vector pDEST12.2.

FIG. 8: Map of pENTR-SCS0010-6HIS.

FIG. 9: Map of pEAK2d-SCS0010-6HIS.

FIG. 10: Map of pDEST12.2-SCS0010-6HIS.

FIG. 11: Alignment between TMEM9 and SCS0010.

FIG. 12: SMART domains of TMEM9 and SCS0010.

### DETAILED DESCRIPTION OF THE INVENTION

According to a first aspect of the present invention there is provided an isolated polypeptide having IL-8-like/TMEM9 activity selected from the group consisting of:
a) the amino acid sequence as recited in SEQ ID NO: 2;
b) the mature form of the polypeptide whose sequence is recited in SEQ ID NO: 2 (SEQ ID NO:4 or SEQ ID NO: 7);
c) the histidine tagged form of the polypeptide whose sequence is recited in SEQ ID NO:4 (SEQ ID NO:5)
d) a variant of the amino acid sequence recited in SEQ ID NO: 2, wherein any amino acid specified in the chosen sequence is non-conservatively substituted, provided that no more than 15% of the amino acid residues in the sequence are so changed;
e) an active fragment, precursor, salt, or derivative of the amino acid sequences given in a) to c).

The novel polypeptide described herein was identified using IL-8-like proteins as query sequences and the final annotation was attributed on the basis of amino acid sequence homology. The novel polypeptide was also identified as a splice variant of an integral transmembrane protein 9 (TMEM9; see examples).

The totality of amino acid sequences obtained by translating the known ORFs in the human genome were challenged using this consensus sequence, and the positive hits were further screened for the presence of predicted specific structural and functional “signatures” that are distinctive of a polypeptide of this nature, and finally selected by comparing sequence features with known IL-8-like polypeptides. Therefore, the novel polypeptides of the invention can be predicted to have IL-8-like and TMEM9 activities.

The terms “active” and “activity” refer to the IL-8-like/TMEM9 properties predicted for the IL-8-like/TMEM9 polypeptide whose amino acid sequence is presented in SEQ ID NO: 2 in the present application. IL-8 and associated proteins have chemotactic activities that attract inflammatory cells, fibroblasts and keratinocytes into wound sites; they act as mitogens to stimulate cellular proliferation; cytokines can stimulate angiogenesis, the ingrowth of new blood vessels into the wound; they have a profound effect on the production and degradation of the ECM; they also influence the synthesis of other cytokines and growth factors by neighbouring cells. Ability to function as an IL-8-like chemokine may be measured using an assay kit such as the Human IL-8 ELISA (IBL, Hamburg) which can detect IL-8 concentrations as low as 70 pg/ml. TMEM9 is described in the examples.

In a second aspect, the invention provides a purified nucleic acid molecule which encodes a polypeptide of the first aspect of the invention. Preferably, the purified nucleic acid molecule has the nucleic acid sequence as recited in SEQ ID NO: 1 (encoding the IL-8-like/TMEM9 polypeptide whose amino acid sequence is recited in SEQ ID NO:2) or SEQ ID NO:3 (encoding the mature form of this polypeptide, whose amino acid sequence is recited in SEQ ID NO:4) or SEQ ID NO:6 (encoding the mature form of this polypeptide, whose amino acid sequence is recited in SEQ ID NO:7). The preferred mature polynucleotide sequence is SEQ ID NO: 3 encoding the preferred mature polypeptide form which is recited in SEQ ID NO: 4.

In a third aspect, the invention provides a purified nucleic acid molecule which hydridizes under high stringency conditions with a nucleic acid molecule of the second aspect of the invention.

In a fourth aspect, the invention provides a vector, such as an expression vector, that contains a nucleic acid molecule of the second or third aspect of the invention. In a fifth aspect, the invention provides a host cell transformed with a vector of the fourth aspect of the invention.

In a sixth aspect, the invention provides a ligand which binds specifically to, and which preferably inhibits the IL-8-like/TMEM9 activity of a polypeptide of the first aspect of the invention. Ligands to a polypeptide according to the invention may come in various forms, including natural or modified substrates, enzymes, receptors, small organic molecules such as small natural or synthetic organic molecules of up to 2000 Da, preferably 800 Da or less, peptidomimetics, inorganic molecules, peptides, polypeptides, antibodies, structural or functional mimetics of the aforementioned.

In a seventh aspect, the invention provides a compound that is effective to alter the expression of a natural gene which encodes a polypeptide of the first aspect of the invention or to regulate the activity of a polypeptide of the first aspect of the invention.

A compound of the seventh aspect of the invention may either increase (agonise) or decrease (antagonise) the level of expression of the gene or the activity of the polypeptide. Importantly, the identification of the function of the IL-8-like/TMEM9 polypeptide of the invention allows for the design of screening methods capable of identifying compounds that are effective in the treatment and/or diagnosis of disease.

In an eighth aspect, the invention provides a polypeptide of the first aspect of the invention, or a nucleic acid molecule of the second or third aspect of the invention, or a vector of the fourth aspect of the invention, or a host cell of the fifth aspect of the invention, or a ligand of the sixth aspect of the invention, or a compound of the seventh aspect of the invention, for use in therapy or diagnosis. These molecules may also be used in the manufacture of a medicament for the prevention and treatment of diseases and conditions in which IL-8-like polypeptides are implicated such as immune disorders, metastasis, transplant rejection, angiogenesis, Alzheimer's disease, atherosclerosis, sublethal endotoxaemia, septic shock, microbial infection of the amniotic cavity, Jarish-Herxheimer reaction of relapsing fever, infectious diseases of the central nervous system, acute pancreatitis, ulcerative colitis, empyaema, haemolytic uraemic syndrome, meningococcal disease, gastric infection, pertussis, peritonitis, psoriasis, rheumatoid arthritis, sepsis, asthma and glomerulonephritis.

In a ninth aspect, the invention provides a method of diagnosing a disease in a patient, comprising assessing the level of expression of a natural gene encoding a polypeptide of the first aspect of the invention or the activity of a polypeptide of the first aspect of the invention in tissue from said patient and comparing said level of expression or activity to a control level, wherein a level that is different to said control level is indicative of disease. Such a method will preferably be carried out in vitro. Similar methods may be used for monitoring the therapeutic treatment of disease in a patient, wherein altering the level of expression or activity of a polypeptide or nucleic acid molecule over the period of time towards a control level is indicative of regression of disease.

A preferred method for detecting polypeptides of the first aspect of the invention comprises the steps of: (a) contacting a ligand, such as an antibody, of the sixth aspect of the invention with a biological sample under conditions suitable for the formation of a ligand-polypeptide complex; and (b) detecting said complex.

A number of different such methods according to the ninth aspect of the invention exist, as the skilled reader will be aware, such as methods of nucleic acid hybridization with short probes, point mutation analysis, polymerase chain reaction (PCR) amplification and methods using antibodies to detect aberrant protein levels. Similar methods may be used on a short or long term basis to allow therapeutic treatment of a disease to be monitored in a patient. The invention also provides kits that are useful in these methods for diagnosing disease.

In a tenth aspect, the invention provides for the use of a polypeptide of the first aspect of the invention as an IL-8-like/TMEM9 protein. Suitable uses include use as a regulator of cellular growth, metabolism or differentiation, use as part of a receptor/ligand pair and use as a diagnostic marker for a physiological or pathological condition selected from the list given above.

In an eleventh aspect, the invention provides a pharmaceutical composition comprising a polypeptide of the first aspect of the invention, or a nucleic acid molecule of the second or third aspect of the invention, or a vector of the fourth aspect of the invention, or a host cell of the fifth aspect of the invention, or a ligand of the sixth aspect of the invention, or a compound of the seventh aspect of the invention, in conjunction with a pharmaceutically-acceptable carrier.

In a twelfth aspect, the present invention provides a polypeptide of the first aspect of the invention, or a nucleic acid molecule of the second or third aspect of the invention, or a vector of the fourth aspect of the invention, or a host cell of the fifth aspect of the invention, or a ligand of the sixth aspect of the invention, or a compound of the seventh aspect of the invention, for use in the manufacture of a medicament for the diagnosis or treatment of a disease or condition in which IL-8-like/TMEM9 polypeptides are implicated such as immune disorders, metastasis, transplant rejection, angiogenesis, Alzheimer's disease, atherosclerosis, sublethal endotoxaemia, septic shock, microbial infection of the amniotic cavity, Jarish-Herxheimer reaction of relapsing fever, infectious diseases of the central nervous system, acute pancreatitis, ulcerative colitis, empyaema, haemolytic uraemic syndrome, meningococcal disease, gastric infection, pertussis, peritonitis, psoriasis, rheumatoid arthritis, sepsis, asthma and glomerulonephritis.

In a thirteenth aspect, the invention provides a method of treating a disease in a patient comprising administering to the patient a polypeptide of the first aspect of the invention, or a nucleic acid molecule of the second or third aspect of the invention, or a vector of the fourth aspect of the invention, or a host cell of the fifth aspect of the invention, or a ligand of the sixth aspect of the invention, or a compound of the seventh aspect of the invention.

For diseases in which the expression of a natural gene encoding a polypeptide of the first aspect of the invention, or in which the activity of a polypeptide of the first aspect of the invention, is lower in a diseased patient when compared to the level of expression or activity in a healthy patient, the polypeptide, nucleic acid molecule, ligand or compound administered to the patient should be an agonist. Conversely, for diseases in which the expression of the natural gene or activity of the polypeptide is higher in a diseased patient when compared to the level of expression or activity in a healthy patient, the polypeptide, nucleic acid molecule, ligand or compound administered to the patient should be an antagonist. Examples of such antagonists include antisense nucleic acid molecules, ribozymes and ligands, such as antibodies.

In a fourteenth aspect, the invention provides transgenic or knockout non-human animals that have been transformed to express higher, lower or absent levels of a polypeptide of the first aspect of the invention. Such transgenic animals are very useful models for the study of disease and may also be used in screening regimes for the identification of compounds that are effective in the treatment or diagnosis of such a disease.

A summary of standard techniques and procedures which may be employed in order to utilise the invention is given below. It will be understood that this invention is not limited to the particular methodology, protocols, cell lines, vectors and reagents described. It is also to be understood that the terminology used herein is for the purpose of describing particular embodiments only and it is not intended that this terminology should limit the scope of the present invention. The extent of the invention is limited only by the terms of the appended claims.

Standard abbreviations for nucleotides and amino acids are used in this specification.

The practice of the present invention will employ, unless otherwise indicated, conventional techniques of molecular biology, microbiology, recombinant DNA technology and immunology, which are within the skill of the those working in the art.

Such techniques are explained fully in the literature. Examples of particularly suitable texts for consultation include the following: Sambrook Molecular Cloning; A Laboratory Manual, Second Edition (1989); DNA Cloning, Volumes I and II (D. N Glover ed. 1985); Oligonucleotide Synthesis (M. J. Gait ed. 1984); Nucleic Acid Hybridization (B. D. Hames & S. J. Higgins eds. 1984); Transcription and Translation (B. D. Hames & S. J. Higgins eds. 1984); Animal Cell Culture (R. I. Freshney ed. 1986); Immobilized Cells and Enzymes (IRL Press, 1986); B. Perbal, A Practical Guide to Molecular Cloning (1984); the Methods in Enzymology series (Academic Press, Inc.), especially volumes 154 & 155; Gene Transfer Vectors for Mammalian Cells (J. H. Miller and M. P. Calos eds. 1987, Cold Spring Harbor Laboratory); Immunochemical Methods in Cell and Molecular Biology (Mayer and Walker, eds. 1987, Academic Press, London); Scopes, (1987) Protein Purification: Principles and Practice, Second Edition (Springer Verlag, N. Y.); and Handbook of Experimental Immunology, Volumes I-IV (D. M. Weir and C. C. Blackwell eds. 1986).

The first aspect of the invention includes variants of the amino acid sequence recited in SEQ ID NO: 2 or SEQ ID NO:4 or SEQ ID NO: 5 or SEQ ID NO: 6, wherein any amino acid specified in the chosen sequence is non-conservatively substituted, provided that no more than 15% of the amino acid residues in the sequence are so changed. Protein sequences having the indicated number of non-conservative substitutions can be identified using commonly available bioinformatic tools (Mulder N J and Apweiler R, 2002; Rehm B H, 2001).

In addition to such sequences, a series of polypeptides forms part of the disclosure of the invention. Being I L-8-like polypeptides known to go through maturation processes including the proteolytic removal of N-terminal sequences (by signal peptidases and other proteolytic enzymes), the present application also claims the mature forms of the polypeptide whose sequence is recited in SEQ ID NO: 2. The sequences of these polypeptides are recited in SEQ ID NO: 4 or SEQ ID NO: 7. The preferred mature form is recited in SEQ ID NO: 4. Mature forms are intended to include any polypeptide showing IL-8-like/TMEM9 activity and resulting from in vivo (by the expressing cells or animals) or in vitro (by modifying the purified polypeptides with specific enzymes) post-translational maturation processes. Other alternative mature forms can also result from the addition of chemical groups such as sugars or phosphates.

The present application also claims the histidine tagged form of the polypeptide whose sequence is recited in SEQ ID NO: 4. The sequence of this polypeptide is recited in SEQ ID NO: 5.

Other claimed polypeptides are the active variants of the amino acid sequences given by SEQ ID NO: 2, SEQ ID NO: 4 or SEQ ID NO: 5 or SEQ ID NO: 7, wherein any amino acid specified in the chosen sequence is non-conservatively substituted, provided that no more than 15%, preferably no more that 10%, 5%, 3%, or 1%, of the amino acid residues in the sequence are so changed. The indicated percentage has to be measured over the novel amino acid sequences disclosed.

In accordance with the present invention, any substitution should be preferably a “conservative” or “safe” substitution, which is commonly defined a substitution introducing an amino acids having sufficiently similar chemical properties (e.g. a basic, positively charged amino acid should be replaced by another basic, positively charged amino acid), in order to preserve the structure and the biological function of the molecule.

The literature provide many models on which the selection of conservative amino acids substitutions can be performed on the basis of statistical and physico-chemical studies on the sequence and/or the structure of proteins (Rogov S I and Nekrasov A N, 2001). Protein design experiments have shown that the use of specific subsets of amino acids can produce foldable and active proteins, helping in the classification of amino acid “synonymous” substitutions which can be more easily accommodated in protein structure, and which can be used to detect functional and structural homologs and paralogs (Murphy L R et al., 2000). The groups of synonymous amino acids and the groups of more preferred synonymous amino acids are shown in Table I.

Active variants having comparable, or even improved, activity with respect of corresponding IL-8-like/TMEM9 polypeptides may result from conventional mutagenesis technique of the encoding DNA, from combinatorial technologies at the level of encoding DNA sequence (such as DNA shuffling, phage display/selection), or from computer-aided design studies, followed by the validation for the desired activities as described in the prior art.

Specific, non-conservative mutations can be also introduced in the polypeptides of the invention with different purposes. Mutations reducing the affinity of the IL-8-like/TMEM9 polypeptide may increase its ability to be reused and recycled, potentially increasing its therapeutic potency (Robinson C R, 2002). Immunogenic epitopes eventually present in the polypeptides of the invention can be exploited for developing vaccines (Stevanovic S, 2002), or eliminated by modifying their sequence following known methods for selecting mutations for increasing protein stability, and correcting them (van den Burg B and Eijsink V, 2002; WO 02/05146, WO 00/34317, WO 98/52976).

Further alternative polypeptides of the invention are active fragments, precursors, salts, or functionally-equivalent derivatives of the amino acid sequences described above. Fragments should present deletions of terminal or internal amino acids not altering their function, and should involve generally a few amino acids, e.g., under ten, and preferably under three, without removing or displacing amino acids which are critical to the functional conformation of the proteins. Small fragments may form an antigenic determinant.

The “precursors” are compounds which can be converted into the compounds of present invention by metabolic and enzymatic processing prior or after the administration to the cells or to the body.

The term “salts” herein refers to both salts of carboxyl groups and to acid addition salts of amino groups of the polypeptides of the present invention. Salts of a carboxyl group may be formed by means known in the art and include inorganic salts, for example, sodium, calcium, ammonium, ferric or zinc salts, and the like, and salts with organic bases as those formed, for example, with amines, such as triethanolamine, arginine or lysine, piperidine, procaine and the like. Acid addition salts include, for example, salts with mineral acids such as, for example, hydrochloric acid or sulfuric acid, and salts with organic acids such as, for example, acetic acid or oxalic acid. Any of such salts should have substantially similar activity to the peptides and polypeptides of the invention or their analogs.

The term “derivatives” as herein used refers to derivatives which can be prepared from the functional groups present on the lateral chains of the amino acid moieties or on the amino- or carboxy-terminal groups according to known methods. Such molecules can result also from other modifications which do not normally alter primary sequence, for example in vivo or in vitro chemical derivativization of polypeptides (acetylation or carboxylation), those made by modifying the pattern of phosphorylation (introduction of phosphotyrosine, phosphoserine, or phosphothreonine residues) or glycosylation (by exposing the polypeptide to mammalian glycosylating enzymes) of a peptide during its synthesis and processing or in further processing steps. Alternatively, derivatives may include esters or aliphatic amides of the carboxyl-groups and N-acyl derivatives of free amino groups or O-acyl derivatives of free hydroxyl-groups and are formed with acyl-groups as for example alcanoyl- or aryl-groups.

The generation of the derivatives may involve a site-directed modification of an appropriate residue, in an internal or terminal position. The residues used for attachment should they have a side-chain amenable for polymer attachment (i.e., the side chain of an amino acid bearing a functional group, e.g., lysine, aspartic acid, glutamic acid, cysteine, histidine, etc.). Alternatively, a residue having a side chain amenable for polymer attachment can replace an amino acid of the polypeptide, or can be added in an internal or terminal position of the polypeptide. Also, the side chains of the genetically encoded amino acids can be chemically modified for polymer attachment, or unnatural amino acids with appropriate side chain functional groups can be employed. The preferred method of attachment employs a combination of peptide synthesis and chemical ligation. Advantageously, the attachment of a water-soluble polymer will be through a biodegradable linker, especially at the amino-terminal region of a protein. Such modification acts to provide the protein in a precursor (or “pro-drug”) form, that, upon degradation of the linker releases the protein without polymer modification.

Polymer attachment may be not only to the side chain of the amino acid naturally occurring in a specific position of the antagonist or to the side chain of a natural or unnatural amino acid that replaces the amino acid naturally occurring in a specific position of the antagonist, but also to a carbohydrate or other moiety that is attached to the side chain of the amino acid at the target position. Rare or unnatural amino acids can be also introduced by expressing the protein in specifically engineered bacterial strains (Bock A, 2001).

All the above indicated variants can be natural, being identified in organisms other than humans, or artificial, being prepared by chemical synthesis, by site-directed mutagenesis techniques, or any other known technique suitable thereof, which provide a finite set of substantially corresponding mutated or shortened peptides or polypeptides which can be routinely obtained and tested by one of ordinary skill in the art using the teachings presented in the prior art.

The novel amino acid sequences disclosed in the present patent application can be used to provide different kind of reagents and molecules. Examples of these compounds are binding proteins or antibodies that can be identified using their full sequence or specific fragments, such as antigenic determinants. Peptide libraries can be used in known methods (Tribbick G, 2002) for screening and characterizing antibodies or other proteins binding the claimed amino acid sequences, and for identifying alternative forms of the polypeptides of the invention having similar binding properties.

The present patent application discloses also fusion proteins comprising any of the polypeptides described above. These polypeptides should contain protein sequence heterologous to the one disclosed in the present patent application, without significantly impairing the IL-8-like/TMEM9 activity of the polypeptide and possibly providing additional properties. Examples of such properties are an easier purification procedure, a longer lasting half-life in body fluids, an additional binding moiety, the maturation by means of an endoproteolytic digestion, or extracellular localization. This latter feature is of particular importance for defining a specific group of fusion or chimeric proteins included in the above definition since it allows the claimed molecules to be localized in the space where not only isolation and purification of these polypeptides is facilitated, but also where generally IL-8-like/TMEM9 polypeptides and their receptor interact.

Design of the moieties, ligands, and linkers, as well methods and strategies for the construction, purification, detection and use of fusion proteins are disclosed in the literature (Nilsson J et al., 1997; Methods Enzymol, Vol. 326-328, Academic Press, 2000). The preferred one or more protein sequences which can be comprised in the fusion proteins belong to these protein sequences: membrane-bound protein, immunoglobulin constant region, multimerization domains, extracellular proteins, signal peptide-containing proteins, export signal-containing proteins. Features of these sequences and their specific uses are disclosed in a detailed manner, for example, for albumin fusion proteins (WO 01/77137), fusion proteins including multimerization domain (WO 01/02440, WO 00/24782), immunoconjugates (Garnett M C, 2001), or fusion protein providing additional sequences which can be used for purifying the recombinant products by affinity chromatography (Constans A, 2002; Burgess R R and Thompson N E, 2002; Lowe C R et al., 2001; J. Bioch. Biophy. Meth., vol. 49 (1-3), 2001; Sheibani N, 1999).

The polypeptides of the invention can be used to generate and characterize ligands binding specifically to them. These molecules can be natural or artificial, very different from the chemical point of view (binding proteins, antibodies, molecularly imprinted polymers), and can be produced by applying the teachings in the art (WO 02/4938; Kuroiwa Y et al., 2002; Haupt K, 2002; van Dijk M A and van de Winkel J G, 2001; Gavilondo J V and Larrick J W, 2000). Such ligands can antagonize or inhibit the IL-8-like activity of the polypeptide against which they have been generated. In particular, common and efficient ligands are represented by extracellular domain of a membrane-bound protein or antibodies, which can be in the form monoclonal, polyclonal, humanized antibody, or an antigen binding fragment.

The polypeptides and the polypeptide-based derived reagents described above can be in alternative forms, according to the desired method of use and/or production, such as active conjugates or complexes with a molecule chosen amongst radioactive labels, fluorescent labels, biotin, or cytotoxic agents.

Specific molecules, such as peptide mimetics, can be also designed on the sequence and/or the structure of a polypeptide of the invention. Peptide mimetics (also called peptidomimetics) are peptides chemically modified at the level of amino acid side chains, of amino acid chirality, and/or of the peptide backbone. These alterations are intended to provide agonists or antagonists of the polypeptides of the invention with improved preparation, potency and/or pharmacokinetics features.

For example, when the peptide is susceptible to cleavage by peptidases following injection into the subject is a problem, replacement of a particularly sensitive peptide bond with a non-cleavable peptide mimetic can provide a peptide more stable and thus more useful as a therapeutic. Similarly, the replacement of an L-amino acid residue is a standard way of rendering the peptide less sensitive to proteolysis, and finally more similar to organic compounds other than peptides. Also useful are amino-terminal blocking groups such as t-butyloxycarbonyl, acetyl, theyl, succinyl, methoxysuccinyl, suberyl, adipyl, azelayl, dansyl, benzyloxycarbonyl, fluorenylmethoxycarbonyl, methoxyazelayl, methoxyadipyl, methoxysuberyl, and 2,4-dinitrophenyl. Many other modifications providing increased potency, prolonged activity, easiness of purification, and/or increased half-life are disclosed in the prior art (WO 02/10195; Villain M et al., 2001).

Preferred alternative, synonymous groups for amino acids derivatives included in peptide mimetics are those defined in Table II. A non-exhaustive list of amino acid derivatives also include aminoisobutyric acid (Aib), hydroxyproline (Hyp), 1,2,3,4-tetrahydro-isoquinoline-3-COOH, indoline-2carboxylic acid, 4-difluoro-proline, L-thiazolidine-4-carboxylic acid, L-homoproline, 3,4-dehydro-proline, 3,4-dihydroxy-phenylalanine, cyclohexyl-glycine, and phenylglycine.

By “amino acid derivative” is intended an amino acid or amino acid-like chemical entity other than one of the 20 genetically encoded naturally occurring amino acids. In particular, the amino acid derivative may contain substituted or non-substituted, linear, branched, or cyclic alkyl moieties, and may include one or more heteroatoms. The amino acid derivatives can be made de novo or obtained from commercial sources (Calbiochem-Novabiochem A G, Switzerland; Bachem, USA).

Various methodologies for incorporating unnatural amino acids derivatives into proteins, using both in vitro and in vivo translation systems, to probe and/or improve protein structure and function are disclosed in the literature (Dougherty D A, 2000).

Techniques for the synthesis and the development of peptide mimetics, as well as non-peptide mimetics, are also well known in the art (Golebiowski A et al., 2001; Hruby V J and Balse P M, 2000; Sawyer T K, in “Structure Based Drug Design”, edited by Veerapandian P, Marcel Dekker Inc., pg. 557-663, 1997).

Another object of the present invention are isolated nucleic acids encoding for the polypeptides of the invention having Il-8-like/TMEM9 activity, the polypeptides binding to an antibody or a binding protein generated against them, the corresponding fusion proteins, or mutants having antagonistic activity as disclosed above. Preferably, these nucleic acids should comprise a DNA sequence selected from the group consisting of SEQ ID NO: 1 or SEQ ID NO: 3 or SEQ ID NO: 6, or the complement of said DNA sequences.

Alternatively, the nucleic acids of the invention should hybridize under high stringency conditions, or exhibit at least about 85% identity over a stretch of at least about 30 nucleotides, with a nucleic acid consisting of SEQ ID NO: 1 or SEQ ID NO: 3 or SEQ ID NO: 6, or be a complement of said DNA sequence.

The wording “high stringency conditions” refers to conditions in a hybridization reaction that facilitate the association of very similar molecules and consist in the overnight incubation at 60-65° C. in a solution comprising 50% formamide, 5× SSC (150 mM NaCl, 15 mM trisodium citrate), 50 mM sodium phosphate (pH 7.6), 5× Denhardt's solution, 10% dextran sulphate, and 20 microgram/ml denatured, sheared salmon sperm DNA, followed by washing the filters in 0.1× SSC at the same temperature.

These nucleic acids, including nucleotide sequences substantially the same, can be comprised in plasmids, vectors and any other DNA construct which can be used for maintaining, modifying, introducing, or expressing the encoding polypeptide. In particular, vectors wherein said nucleic acid molecule is operatively linked to expression control sequences can allow expression in prokaryotic or eukaryotic host cells of the encoded polypeptide.

The wording “nucleotide sequences substantially the same” includes all other nucleic acid sequences which, by virtue of the degeneracy of the genetic code, also code for the given amino acid sequences. In this sense, the literature provides indications on preferred or optimized codons for recombinant expression (Kane J F et al., 1995).

The nucleic acids and the vectors can be introduced into cells with different purposes, generating transgenic cells and organisms. A process for producing cells capable of expressing a polypeptide of the invention comprises genetically engineering cells with such vectors and nucleic acids.

In particular, host cells (e.g. bacterial cells) can be modified by transformation for allowing the transient or stable expression of the polypeptides encoded by the nucleic acids and the vectors of the invention. Alternatively, said molecules can be used to generate transgenic animal cells or non-human animals (by non-/homologous recombination or by any other method allowing their stable integration and maintenance), having enhanced or reduced expression levels of the polypeptides of the invention, when the level is compared with the normal expression levels. Such precise modifications can be obtained by making use of the nucleic acids of the inventions and of technologies associated, for example, to gene therapy (Meth. Enzymol., vol. 346, 2002) or to site-specific recombinases (Kolb A F, 2002). Model systems based on the IL-8-like/TMEM9 polypeptides disclosed in the present patent application for the systematic study of their function can be also generated by gene targeting into human cell lines (Bunz F, 2002).

Gene silencing approaches may also be undertaken to down-regulate endogenous expression of a gene encoding a polypeptide of the invention. RNA interference (RNAi) (Elbashir, S M et al., Nature 2001, 411, 494-498) is one method of sequence specific post-transcriptional gene silencing that may be employed. Short dsRNA oligonucleotides are synthesised in vitro and introduced into a cell. The sequence specific binding of these dsRNA oligonucleotides triggers the degradation of target mRNA, reducing or ablating target protein expression.

Efficacy of the gene silencing approaches assessed above may be assessed through the measurement of polypeptide expression (for example, by Western blotting), and at the RNA level using TaqMan-based methodologies.

The polypeptides of the invention can be prepared by any method known in the art, including recombinant DNA-related technologies, and chemical synthesis technologies. In particular, a method for making a polypeptide of the invention may comprise culturing a host or transgenic cell as described above under conditions in which the nucleic acid or vector is expressed, and recovering the polypeptide encoded by said nucleic acid or vector from the culture. For example, when the vector expresses the polypeptide as a fusion protein with an extracellular or signal-peptide containing proteins, the recombinant product can be secreted in the extracellular space, and can be more easily collected and purified from cultured cells in view of further processing or, alternatively, the cells can be directly used or administered.

The DNA sequence coding for the proteins of the invention can be inserted and ligated into a suitable episomal or non-/homologously integrating vectors, which can be introduced in the appropriate host cells by any suitable means (transformation, transfection, conjugation, protoplast fusion, electroporation, calcium phosphate-precipitation, direct microinjection, etc.). Factors of importance in selecting a particular plasmid or viral vector include: the ease with which recipient cells that contain the vector, may be recognized and selected from those recipient cells which do not contain the vector; the number of copies of the vector which are desired in a particular host; and whether it is desirable to be able to “shuttle” the vector between host cells of different species.

The vectors should allow the expression of the isolated or fusion protein including the polypeptide of the invention in the prokaryotic or eukaryotic host cells under the control of transcriptional initiation/termination regulatory sequences, which are chosen to be constitutively active or inducible in said cell. A cell line substantially enriched in such cells can be then isolated to provide a stable cell line.

For eukaryotic hosts (e.g. yeasts, insect, plant, or mammalian cells), different transcriptional and translational regulatory sequences may be employed, depending on the nature of the host. They may be derived form viral sources, such as adenovirus, bovine papilloma virus, Simian virus or the like, where the regulatory signals are associated with a particular gene which has a high level of expression. Examples are the TK promoter of the Herpes virus, the SV40 early promoter, the yeast gal4 gene promoter, etc. Transcriptional initiation regulatory signals may be selected which allow for repression and activation, so that expression of the genes can be modulated. The cells stably transformed by the introduced DNA can be selected by introducing one or more markers allowing the selection of host cells which contain the expression vector. The marker may also provide for phototrophy to an auxotropic host, biocide resistance, e.g. antibiotics, or heavy metals such as copper, or the like. The selectable marker gene can either be directly linked to the DNA gene sequences to be expressed, or introduced into the same cell by co-transfection.

Host cells may be either prokaryotic or eukaryotic. Preferred are eukaryotic hosts, e.g. mammalian cells, such as human, monkey, mouse, and Chinese Hamster Ovary (CHO) cells, because they provide post-translational modifications to proteins, including correct folding and glycosylation. Also yeast cells can carry out post-translational peptide modifications including glycosylation. A number of recombinant DNA strategies exist which utilize strong promoter sequences and high copy number of plasmids which can be utilized for production of the desired proteins in yeast. Yeast recognizes leader sequences in cloned mammalian gene products and secretes peptides bearing leader sequences (i.e., pre-peptides).

The above mentioned embodiments of the invention can be achieved by combining the disclosure provided by the present patent application on the sequence of novel IL-8-like/TMEM9 polypeptides with the knowledge of common molecular biology techniques.

Many books and reviews provides teachings on how to clone and produce recombinant proteins using vectors and Prokaryotic or Eukaryotic host cells, such as some titles in the series “A Practical Approach” published by Oxford University Press (“DNA Cloning 2: Expression Systems”, 1995; “DNA Cloning 4: Mammalian Systems”, 1996; “Protein Expression”, 1999; “Protein Purification Techniques”, 2001).

Moreover, updated and more focused literature provides an overview of the technologies for expressing polypeptides in a high-throughput manner (Chambers S P, 2002; Coleman T A, et al., 1997), of the cell systems and the processes used industrially for the large-scale production of recombinant proteins having therapeutic applications (Andersen D C and Krummen L, 2002, Chu L and Robinson D K, 2001), and of alternative eukaryotic expression systems for expressing the polypeptide of interest, which may have considerable potential for the economic production of the desired protein, such the ones based on transgenic plants (Giddings G, 2001) or the yeast Pichia pastoris (Lin Cereghino G P et al., 2002). Recombinant protein products can be rapidly monitored with various analytical technologies during purification to verify the amount and the quantity of the expressed polypeptides (Baker K N et al., 2002), as well as to check if there is problem of bioequivalence and immunogenicity (Schellekens H, 2002; Gendel S M, 2002).

Totally synthetic IL-8-like polypeptides are disclosed in the literature and many examples of chemical synthesis technologies, which can be effectively applied for the IL-8-like/TMEM9 polypeptides of the invention given their short length, are available in the literature, as solid phase or liquid phase synthesis technologies. For example, the amino acid corresponding to the carboxy-terminus of the peptide to be synthesized is bound to a support which is insoluble in organic solvents, and by alternate repetition of reactions, one wherein amino acids with their amino groups and side chain functional groups protected with appropriate protective groups are condensed one by one in order from the carboxy-terminus to the amino-terminus, and one where the amino acids bound to the resin or the protective group of the amino groups of the peptides are released, the peptide chain is thus extended in this manner. Solid phase synthesis methods are largely classified by the tBoc method and the Fmoc method, depending on the type of protective group used. Typically used protective groups include tBoc (t-butoxycarbonyl), cl-z (2-chlorobenzyloxycarbonyl), Br-Z (2-bromobenzyloxycarbonyl), Bzl (benzyl), Fmoc (9-fluorenylmethoxycarbonyl), Mbh (4,4′-dimethoxydibenzhydryl), Mtr (4-methoxy-2,3,6-trimethylbenzenesulphonyl), Trt (trityl), Tos (tosyl), Z (benzyloxycarbonyl) and C12-Bzl (2,6-dichlorobenzyl) for the amino groups; NO₂ (nitro) and Pmc (2,2,5,7,8-pentamethylchromane-6-sulphonyl) for the guanidino groups); and tBu (t-butyl) for the hydroxyl groups). After synthesis of the desired peptide, it is subjected to the de-protection reaction and cut out from the solid support. Such peptide cutting reaction may be carried with hydrogen fluoride or tri-fluoromethane sulfonic acid for the Boc method, and with TFA for the Fmoc method.

The purification of the polypeptides of the invention can be carried out by any one of the methods known for this purpose, i.e. any conventional procedure involving extraction, precipitation, chromatography, electrophoresis, or the like. A further purification procedure that may be used in preference for purifying the protein of the invention is affinity chromatography using monoclonal antibodies or affinity groups, which bind the target protein and which are produced and immobilized on a gel matrix contained within a column. Impure preparations containing the proteins are passed through the column. The protein will be bound to the column by heparin or by the specific antibody while the impurities will pass through. After washing, the protein is eluted from the gel by a change in pH or ionic strength. Alternatively, HPLC (High Performance Liquid Chromatography) can be used. The elution can be carried using a water-acetonitrile-based solvent commonly employed for protein purification.

The disclosure of the novel polypeptides of the invention, and the reagents disclosed in connection to them (antibodies, nucleic acids, cells) allows also to screen and characterize compounds that enhance or reduce their expression level into a cell or in an animal.

“Oligonucleotides” refers to either a single stranded polydeoxynucleotide or two complementary polydeoxynucleotide strands which may be chemically synthesized. Such synthetic oligonucleotides have no 5′ phosphate and thus will not ligate to another oligonucleotide without adding a phosphate with an ATP in the presence of a kinase. A synthetic oligonucleotide will ligate to a fragment that has not been dephosphorylated.

The invention includes purified preparations of the compounds of the invention (polypeptides, nucleic acids, cells, etc.). Purified preparations, as used herein, refers to the preparations which contain at least 1%, preferably at least 5%, by dry weight of the compounds of the invention.

The present patent application discloses a series of novel IL-8-like/TMEM9 polypeptides and of related reagents having several possible applications. In particular, whenever an increase in the IL-8-like/TMEM9 activity of a polypeptide of the invention is desirable in the therapy or in the prevention of a disease, reagents such as the disclosed IL-8-like/TMEM9 polypeptides, the corresponding fusion proteins and peptide mimetics, the encoding nucleic acids, the expressing cells, or the compounds enhancing their expression can be used.

Therefore, the present invention discloses pharmaceutical compositions for the treatment or prevention of diseases needing an increase in the IL-8-like/TMEM9 activity of a polypeptide of the invention, which contain one of the disclosed IL-8-like/TMEM9 polypeptides, the corresponding fusion proteins and peptide mimetics, the encoding nucleic acids, the expressing cells, or the compounds enhancing their expression, as active ingredient. The process for the preparation of these pharmaceutical compositions comprises combining the disclosed IL-8-like/TMEM9 polypeptides, the corresponding fusion proteins and peptide mimetics, the encoding nucleic acids, the expressing cells, or the compounds enhancing their expression, together with a pharmaceutically acceptable carrier. Methods for the treatment or prevention of diseases needing an increase in the IL-8-like/TMEM9 activity of a polypeptide of the invention, comprise the administration of a therapeutically effective amount of the disclosed IL-8-like/TMEM9 polypeptides, the corresponding fusion proteins and peptide mimetics, the encoding nucleic acids, the expressing cells, or the compounds enhancing their expression.

Amongst the reagents disclosed in the present patent application, the ligands, the antagonists or the compounds reducing the expression or the activity of polypeptides of the invention have several applications, and in particular they can be used in the therapy or in the diagnosis of a disease associated to the excessive IL-8-like/TMEM9 activity of a polypeptide of the invention.

Therefore, the present invention discloses pharmaceutical compositions for the treatment or prevention of diseases associated to the excessive IL-8-like/TMEM9 activity of a polypeptide of the invention, which contain one of the ligands, antagonists, or compounds reducing the expression or the activity of such polypeptides, as active ingredient. The process for the preparation of these pharmaceutical compositions comprises combining the ligand, the antagonist, or the compound, together with a pharmaceutically acceptable carrier. Methods for the treatment or prevention of diseases associated to the excessive IL-8-like/TMEM9 activity of the polypeptide of the invention, comprise the administration of a therapeutically effective amount of the antagonist, the ligand or of the compound.

The pharmaceutical compositions of the invention may contain, in addition to IL-8-like/TMEM9 polypeptide or to the related reagent, suitable pharmaceutically acceptable carriers, biologically compatible vehicles and additives which are suitable for administration to an animal (for example, physiological saline) and eventually comprising auxiliaries (like excipients, stabilizers, adjuvants, or diluents) which facilitate the processing of the active compound into preparations which can be used pharmaceutically.

The pharmaceutical compositions may be formulated in any acceptable way to meet the needs of the mode of administration. For example, of biomaterials, sugar-macromolecule conjugates, hydrogels, polyethylene glycol and other natural or synthetic polymers can be used for improving the active ingredients in terms of drug delivery efficacy. Technologies and models to validate a specific mode of administration are disclosed in literature (Davis B G and Robinson M A, 2002; Gupta P et al., 2002; Luo B and Prestwich G D, 2001; Cleland J L et al., 2001; Pillai O and Panchagnula R, 2001).

Polymers suitable for these purposes are biocompatible, namely, they are non-toxic to biological systems, and many such polymers are known. Such polymers may be hydrophobic or hydrophilic in nature, biodegradable, non-biodegradable, or a combination thereof. These polymers include natural polymers (such as collagen, gelatin, cellulose, hyaluronic acid), as well as synthetic polymers (such as polyesters, polyorthoesters, polyanhydrides). Examples of hydrophobic non-degradable polymers include polydimethyl siloxanes, polyurethanes, polytetrafluoroethylenes, polyethylenes, polyvinyl chlorides, and polymethyl methaerylates. Examples of hydrophilic non-degradable polymers include poly(2-hydroxyethyl methacrylate), polyvinyl alcohol, poly(N-vinyl pyrrolidone), polyalkylenes, polyacrylamide, and copolymers thereof. Preferred polymers comprise as a sequential repeat unit ethylene oxide, such as polyethylene glycol (PEG).

Any accepted mode of administration can be used and determined by those skilled in the art to establish the desired blood levels of the active ingredients. For example, administration may be by various parenteral routes such as subcutaneous, intravenous, intradermal, intramuscular, intraperitoneal, intranasal, transdermal, oral, or buccal routes. The pharmaceutical compositions of the present invention can also be administered in sustained or controlled release dosage forms, including depot injections, osmotic pumps, and the like, for the prolonged administration of the polypeptide at a predetermined rate, preferably in unit dosage forms suitable for single administration of precise dosages.

Parenteral administration can be by bolus injection or by gradual perfusion over time. Preparations for parenteral administration include sterile aqueous or non-aqueous solutions, suspensions, and emulsions, which may contain auxiliary agents or excipients known in the art, and can be prepared according to routine methods. In addition, suspension of the active compounds as appropriate oily injection suspensions may be administered. Suitable lipophilic solvents or vehicles include fatty oils, for example, sesame oil, or synthetic fatty acid esters, for example, sesame oil, or synthetic fatty acid esters, for example, ethyl oleate or triglycerides. Aqueous injection suspensions that may contain substances increasing the viscosity of the suspension include, for example, sodium carboxymethyl cellulose, sorbitol, and/or dextran. Optionally, the suspension may also contain stabilizers. Pharmaceutical compositions include suitable solutions for administration by injection, and contain from about 0.01 to 99.99 percent, preferably from about 20 to 75 percent of active compound together with the excipient.

The wording “therapeutically effective amount” refers to an amount of the active ingredients that is sufficient to affect the course and the severity of the disease, leading to the reduction or remission of such pathology. The effective amount will depend on the route of administration and the condition of the patient.

The wording “pharmaceutically acceptable” is meant to encompass any carrier, which does not interfere with the effectiveness of the biological activity of the active ingredient and that is not toxic to the host to which is administered. For example, for parenteral administration, the above active ingredients may be formulated in unit dosage form for injection in vehicles such as saline, dextrose solution, serum albumin and Ringer's solution. Carriers can be selected also from starch, cellulose, talc, glucose, lactose, sucrose, gelatin, malt, rice, flour, chalk, silica gel, magnesium stearate, sodium stearate, glycerol monostearate, sodium chloride, dried skim milk, glycerol, propylene glycol, water, ethanol, and the various oils, including those of petroleum, animal, vegetable or synthetic origin (peanut oil, soybean oil, mineral oil, sesame oil).

It is understood that the dosage administered will be dependent upon the age, sex, health, and weight of the recipient, kind of concurrent treatment, if any, frequency of treatment, and the nature of the effect desired. The dosage will be tailored to the individual subject, as is understood and determinable by one of skill in the art. The total dose required for each treatment may be administered by multiple doses or in a single dose. The pharmaceutical composition of the present invention may be administered alone or in conjunction with other therapeutics directed to the condition, or directed to other symptoms of the condition. Usually a daily dosage of active ingredient is comprised between 0.01 to 100 milligrams per kilogram of body weight per day. Ordinarily 1 to 40 milligrams per kilogram per day given in divided doses or in sustained release form is effective to obtain the desired results. Second or subsequent administrations can be performed at a dosage, which is the same, less than, or greater than the initial or previous dose administered to the individual.

Apart from methods having a therapeutic or a production purpose, several other methods can make use of the IL-8-like/TMEM9 polypeptides and of the related reagents disclosed in the present patent application.

In a first example, a method is provided for screening candidate compounds effective to treat a disease related to a IL-8-like/TMEM9 polypeptide of the invention, said method comprising:
(a) contacting host cells expressing such polypeptide, transgenic non-human animals, or transgenic animal cells having enhanced or reduced expression levels of the polypeptide, with a candidate compound and
(b) determining the effect of the compound on the animal or on the cell.

In a second example there is provided a method for identifying a candidate compound as an antagonist/inhibitor or agonist/activator of a polypeptide of the invention, the method comprising:
(a) contacting the polypeptide, the compound, and a mammalian cell or a mammalian cell membrane; and
(b) measuring whether the molecule blocks or enhances the interaction of the polypeptide, or the response that results from such interaction, with the mammalian cell or the mammalian cell membrane.

In a third example, a method for determining the activity and/or the presence of the polypeptide of the invention in a sample, can detect either the polypeptide or the encoding RNA/DNA. Thus, such a method comprises:
(a) providing a protein-containing sample;
(b) contacting said sample with a ligand of the invention; and
(c) determining the presence of said ligand bound to said polypeptide, thereby determining the activity and/or the presence of polypeptide in said sample.

In an alternative, the method comprises:
(a) providing a nucleic acids-containing sample;
(b) contacting said sample with a nucleic acid of the invention; and
(c) determining the hybridization of said nucleic acid with a nucleic acid into the sample, thereby determining the presence of the nucleic acid in the sample.

In this sense, a primer sequence derived from the nucleotide sequence presented in SEQ ID NO: 1 or SEQ ID NO: 3 or SEQ ID NO: 6 can be used as well for determining the presence or the amount of a transcript or of a nucleic acid encoding a polypeptide of invention in a sample by means of Polymerase Chain Reaction amplification.

A further object of the present invention are kits for measuring the activity and/or the presence of IL-8-like/TMEM9 polypeptide of the invention in a sample comprising one or more of the reagents disclosed in the present patent application: an 18-like/TMEM9 polypeptide of the invention, an antagonist, ligand or peptide mimetic, an isolated nucleic acid or the vector, a pharmaceutical composition, an expressing cell, or a compound increasing or decreasing the expression levels.

Such kits can be used for in vitro diagnostic or screenings methods, and their actual composition should be adapted to the specific format of the sample (e.g. biological sample tissue from a patient), and the molecular species to be measured. For example, if it is desired to measure the concentration of the IL-8-like/TMEM9 polypeptide, the kit may contain an antibody and the corresponding protein in a purified form to compare the signal obtained in Western blot. Alternatively, if it is desired to measure the concentration of the transcript for the IL-8-like/TMEM9 polypeptide, the kit may contain a specific nucleic acid probe designed on the corresponding ORF sequence, or may be in the form of nucleic acid array containing such probe. The kits can be also in the form of protein-, peptide mimetic-, or cell-based microarrays (Templin M F et al., 2002; Pellois J P et al., 2002; Blagoev B and Pandey A, 2001), allowing high-throughput proteomics studies, by making use of the proteins, peptide mimetics and cells disclosed in the present patent application.

Therapeutic Uses

The present patent application discloses a SCS0010 polypeptide having several possible applications. In particular, whenever an increase in the IL-8-like/TMEM9 activity of a polypeptide of the invention is desirable in the therapy or in the prevention of a disease, reagents such as the disclosed SCS0010 polypeptide, the corresponding fusion proteins and peptide mimetics, the encoding nucleic acids, the expressing cells, or the compounds enhancing their expression can be used.

Therefore, the present invention discloses pharmaceutical compositions for the treatment or prevention of diseases needing an increase in the IL-8-like/TMEM9 activity of a polypeptide of the invention, which contain one of the disclosed IL-8-like/TMEM9 polypeptides, the corresponding fusion proteins and peptide mimetics, the encoding nucleic acids, the expressing cells, or the compounds enhancing their expression, as active ingredient. The process for the preparation of these pharmaceutical compositions comprises combining the disclosed IL8-like/TMEM9 polypeptides, the corresponding fusion proteins and peptide mimetics, the encoding nucleic acids, the expressing cells, or the compounds enhancing their expression, together with a pharmaceutically acceptable carrier. Methods for the treatment or prevention of diseases needing an increase in the IL-8-like/TMEM9 activity of a polypeptide of the invention, comprise the administration of a therapeutically effective amount of the disclosed IL-8-like/TMEM9 polypeptide, the corresponding fusion proteins and peptide mimetics, the encoding nucleic acids, the expressing cells, or the compounds enhancing their expression.

Amongst the reagents disclosed in the present patent application, the ligands, the antagonists or the compounds reducing the expression or the activity of polypeptides of the invention have several applications, and in particular they can be used in the therapy or in the diagnosis of a disease associated to the excessive IL-8-like/TMEM9 activity of a polypeptide of the invention.

Therefore, the present invention discloses pharmaceutical compositions for the treatment or prevention of diseases associated to the excessive IL-8-like/TMEM9 activity of a polypeptide of the invention, which contain one of the ligands, antagonists, or compounds reducing the expression or the activity of such polypeptides, as active ingredient. The process for the preparation of these pharmaceutical compositions comprises combining the ligand, the antagonist, or the compound, together with a pharmaceutically acceptable carrier. Methods for the treatment or prevention of diseases associated to the excessive IL-8-like/TMEM9 activity of the polypeptide of the invention, comprise the administration of a therapeutically effective amount of the antagonist, the ligand or of the compound.

SCS0010 nucleic acid molecule, polypeptide, and agonists and antagonists thereof can be used to treat, diagnose, ameliorate, or prevent a number of diseases, disorders, or conditions, including those recited herein.

SCS0010 polypeptide agonists and antagonists include those molecules which regulate SCS0010 polypeptide activity and either increase or decrease at least one activity of the mature form of the SCS0010 polypeptide. Agonists or antagonists may be co-factors, such as a protein, peptide, carbohydrate, lipid, or small molecular weight molecule, which interact with SCS0010 polypeptide and thereby regulate its activity.

Potential polypeptide agonists or antagonists include antibodies that react with either soluble (SCS0010) or membrane-bound forms (TMEM9) of TMEM9 polypeptides that comprise part or all of the extracellular domains of the said proteins. Molecules that regulate SCS0010 polypeptide expression typically include nucleic acids encoding SCS0010 polypeptide that can act as anti-sense regulators of expression.

Thus, the present patent application discloses novel IL-8-like/TMEM9 polypeptides and a series of related reagents that may be useful, as active ingredients in pharmaceutical compositions appropriately formulated, in the treatment or prevention of diseases and conditions in which IL-8-like/TMEM9 polypeptides are implicated such as immune disorders, metastasis, transplant rejection, angiogenesis, Alzheimer's disease, atherosclerosis, sublethal endotoxaemia, septic shock, microbial infection of the amniotic cavity, Jarish-Herxheimer reaction of relapsing fever, infectious diseases of the central nervous system, acute pancreatitis, ulcerative colitis, empyaema, haemolytic uraemic syndrome, meningococcal disease, gastric infection, pertussis, peritonitis, psoriasis, rheumatoid arthritis, sepsis, asthma and glomerulonephritis. Some of these uses are supported by experiments mainly derived from the Gene Expression Omnibus (GEO) database: http://www.ncbi.nlm.nih.gov/entrez/query.fcgi?db=geo.

“GEO serves as a public repository for a wide range of high-throughput experimental data. These data include single and dual channel microarray-based experiments measuring mRNA, genomic DNA, and protein abundance, as well as non-array techniques such as serial analysis of gene expression (SAGE), and mass spectrometry proteomic data.”

In the GEO record GDS85 on the temporal transcriptional program of primary fibroblasts in response to serum (dual channel microarray experiment), it has been shown that TMEM9 undergoes a slight inhibition with a peak at approximately 1 hour. It is known that fibroblasts are involved in many diseases including vascular diseases, inflammation, fibrosis, fibrotic disorders, rheumatoid arthritis, Crohn's disease, scleroderma, respiratory infections, asthma, allergic reactions, bronchitis, interstitial lung diseases, diabetic nephropathy, cardiac arrhythmia, cardiac hypertrophy, tumors, ulcers, ophthalmopathy, lipodystrophy, systemic sclerosis, osteoarthropathy, neuropathologies, as well as being implicated in aging, tissue repair and plastic surgery. In addition, in the GEO record GDS60, in an analysis of allergic response to an immunogenic protein of ragweed pollen in lung by the determination of the temporal relationship between gene expression and airway allergen challenge (single channel microarray experiment), it has been shown that upon allergen challenge TMEM9 was consistently reduced (TMEM9 is very slightly expressed) in two samples out of three. In the GEO record GDS350, in a pulmonary fibrosis model (A/J, bleomycin resistant), lungs of intratracheal bleomycin-treated A/J mice were compared with controls (dual channel microarray experiment). This study showed that TMEM9 was slightly inhibited in three samples out of four in the pulmonary fibrosis model compared to control.

In the GEO record GDS61, in lung hypertension recovery, an analysis of vascular remodeling following pulmonary hypertension comparing lungs of normoxic and hypoxic treated animals was performed (single channel microarray experiment). Hypoxia induces vasoconstriction then hypertrophy/hyperplasia of pulmonary vascular smooth muscle and endothelial cell proliferation. It was shown that after 10 hours, TMEM9's expression was consistently reduced in hypoxic treated animals compared to normoxic.

In a review on IL-8, Mukaida N. exposes the evidence demonstrating “that various types of cells can produce a large amount of IL-8/CXCL8 in response to a wide variety of stimuli, including proinflammatory cytokines, microbes and their products, and environmental changes such as hypoxia, reperfusion, and hyperoxia.” (Am J Physiol Lung Cell Mol Physiol. 2003 April; 284(4):L566-77. “Pathophysiological roles of interleukin-8/CXCL8 in pulmonary diseases”). In addition, Mukaida mentions that “numerous observations have established IL-8/CXCL8 as a key mediator in neutrophil-mediated acute inflammation due to its potent actions on neutrophils.” Finally, Mukaida refers to “several lines of evidence indicate that IL-8/CXCL8 has a wide range of actions on various types of cells, including lymphocytes, monocytes, endothelial cells, and fibroblasts, besides neutrophils.” Still, biological functions of IL-8 suggest that it has also crucial roles in various pathological conditions such as arterosclerosis, inflammation, lung pathologies and cancer.

SCS0010 may also be useful in cancer therapy as suggested by the following studies. Kurokawa et al. showed in a PCR-array experiment containing 3072 genes derived from three different cDNA libraries and 298 additional known genes suspected to be involved in hepatocarcinogenesis, that only 7 genes, of which TMEM9, may play a common key role in hepatocarcinogenesis (Kurokawa et al. J Exp Clin Cancer Res. 2004 March; 23(1):135-41. PCR-array gene expression profiling of hepatocellular carcinoma.). In the GEO record GDS88, in an analysis of cell lines used in the National Cancer Institute's screen for anti-cancer drugs (dual channel microarray experiment), it was shown that TMEM9 is slightly inhibited in the melanoma, leukemia and ovarian cancer cell lines compared to a reference pool.

In the GEO record GDS143, in epidermal carcinogenesis, a comparison of premalignant skin lesions (actinic keratosis), normal epidermis and cultured keratinocytes was performed (SAGE experiment). It was shown that TMEM9 is highly expressed in normal epidermis, compared with no expression in cultured keratinocytes and actinic keratosis epidermis.

In the GEO record GDS 103, in cardiomyocyte formation, for the characterization of the transcriptomes of P19 embryonic carcinoma (EC) cells in an undifferentiated, pluripotent state and after induction to differentiate into cardiomyocytes (SAGE experiment), TMEM9 was shown to be expressed in P19 EC cells 3.5 days after plating, compared with EC cells 0.5 days after plating or to undifferentiated P19 EC cells where no TMEM9's expression occurred.

In the GEO record GDS121, a comprehensive analysis of gene expression profiles following estrogen or tamoxifen treatment was performed (SAGE experiment). It was shown that tamoxifen treated ZR75-1 human breast cancer cell line induced the expression of TMEM9 compared with untreated or estrogen treated ZR75-1 cells.

In the GEO record GDS122, in the identification of targets of c-Jun NH82-terminal kinase 2-mediated tumor growth regulation highlighting the importance of JNK2 signaling in regulating cell homeostasis and tumor cell growth (SAGE experiment), it was shown that TMEM9's expression is higher in JNK2 antisense oligonucleotides PC3 treated cells compared with mock treated PC3 cells. IL-8 activity has been shown to be sensitive to the dominant-negative mutants of JNK2 and that coordinate activation of NF-kappaB and JNK is required for strong IL-8 transcription.

Several GEO records also provide TMEM9's expression in a variety of tissues (GDS503, GDS8, GDS393, GDS217, GDS541, GDS546, GDS217, GDS381, GDS102, GDS541, GDS117, GDS541, GDS544, GDS580, GDS551, GDS545, GDS542 and GDS548). Expression of TMEM9 has been shown in kidney (proximal convoluted tubule, cortical collecting duct, cortical thick ascending limb), whole brain, nucleus accumbens, brain ependymoma, brain medulloblastoma, lymph node, mammary epithelium, breast myoepithelial, breast carcinoma, ductal carcinoma (breast), white matter, pediatric cortex, peripheral retina, central retina, peritoneum, colon (primary tumor), gastric cancer, ovarian surface epithelium, primary malignant skin melanoma, prostate carcinoma (advanced tumor). According to the Genecard entry for TMEM9 ( http://bioinformatics.weizmann.ac.il/cards/), TMEM9 is expressed in bone marrow, spleen, thymus, brain, spinal chord, heart, skeletal muscle, liver, pancreas, prostate, kidney and lung.

Expression information is also available in the UniGene Cluster Hs.181444 for TMEM9 (Homo sapiens; http://www.ncbi.nlm.nih.gov/entrez/query.fcgi?db=unigene). According to Unigene, TMEM9 is highly expressed in library 5967 NT0048, which is derived from an adult Homo sapiens nervous tumor. In addition cDNA sources for TMEM9 are:

serious papillary carcinoma, high grade, 2 pooled tumors; heart; moderately-differentiated endometrial adenocarcinoma, 3 pooled tumors; glioblastoma (pooled); poorly differentiated adenocarcinoma with signet ring cell features; poorly-differentiated endometrial adenocarcinoma, 2 pooled tumors; anaplastic oligodendroglioma; lymphoma, follicular mixed small and large cell; well-differentiated endometrial adenocarcinoma, 7 pooled tumors; squamous cell carcinoma, poorly differentiated (4 pooled tumors, including primary and metastatic); pooled; testis; NEUROBLASTOMA COT 50-NORMALIZED; thymus, pooled; uterus; Bone; normal endometrium, mid-secretory phase, cycle day 23; Pituitary; NEUROBLASTOMA COT 25-NORMALIZED; PLACENTA COT 25-NORMALIZED; early stage papillary serous carcinoma; placenta; adenocarcinoma; tumor, 5 pooled (see description); normal nasopharynx; colonic mucosa from 3 patients with Crohn's disease; five pooled sarcomas, including myxoid liposarcoma, solitary fibrous tumor, malignant fibrous histiocytoma, gastrointestinal stromal tumor, and mesothelioma; total brain; Liver and Spleen; Testis; Liver; FETAL LIVER; lung; carcinoid; moderately differentiated adenocarcinoma; 2 pooled Wilms' tumors, one primary and one metastatic to brain; medulloblastoma; CNCAP(3)T-225 cell line; breast; colon; two pooled squamous cell carcinomas; Purified pancreatic islet; head_neck; Peripheral Nervous system; Placenta; Pooled Chondrosarcoma Tumor cells; carcinoma cell line; adenocarcinoma cell line; retinoblastoma; renal cell adenocarcinoma; pooled germ cell tumors; brain; Brain; prostate; squamous cell carcinoma; cervical carcinoma cell line; large cell carcinoma; small cell carcinoma; endometrium, adenocarcinoma cell line; melanotic melanoma; kidney tumor; choriocarcinoma; breast_normal; large cell carcinoma, undifferentiated; nervous_tumor; pancreas; ovary; hypernephroma; embryonic stem cells, WAO1, passage 38; cord blood; glioblastoma with EGFR amplification; testis_normal; from acute myelogenous leukemia; anaplastic oligodendroglioma with 1p/19 q loss; melanocyte; adenocarcinoma, cell line; adrenal cortex carcinoma, cell line; muscle (skeletal); duodenal adenocarcinoma, cell line; retina; epithelium (cell line); hypemephroma, cell line; NEUROBLASTOMA; mucoepidermoid carcinoma; cartilage; hippocampus; multiple sclerosis lesions; Blood; leukocyte; primitive neuroectoderm; NEUROBLASTOMA COT 10-NORMALIZED; epithelioid carcinoma cell line; transitional cell papilloma, cell line; lung_normal; hypothalamus; pooled colon, kidney, stomach; pooled pancreas and spleen; carcinoma, cell line; medulla; pooled lung and spleen; bladder_tumor; pooled brain, lung, testis; parathyroid tumor; optic nerve; ovary (pool of 3); human retina; neuroblastoma; fetal eyes, lens, eye anterior segment, optic nerve, retina, Retina Foveal and Macular, RPE and Choroid; RPE and Choroid; fetal eyes; Retina Foveal and Macular; Ascites Pooled human melanocyte, fetal heart, and pregnant uterus; frontal lobe; FETAL BRAIN; malignant melanoma, metastatic to lymph node; whole brain; 2 pooled tumors (clear cell type); kidney; Stomach; eye; Lung; B-cell, chronic lymphotic leukemia; Primary Lung Cystic Fibrosis Epithelial Cells; mixed; Metastatic Chondrosarcoma; leiomyosarcoma; Chondrosarcoma Grade II; Lung Focal Fibrosis; Chondrosarcoma; Retina; hepatocellular carcinoma, cell line; papillary serous carcinoma; sympathetic trunk; glioblastoma with probably TP53 mutation and without EGFR amplification; dorsal root ganglia; lung carcinoma; mixed (pool of 40 RNAs); myeloma; Cell lines; teratocarcinoma, cell line; insulinoma; germinal center B cell; lens; Aveolar Macrophage; RPE/choroid; Adipose; Chondrosarcoma Cell line; Enchondroma cell line; Subchondral Bone; endometrium; embryo; epididymis; ovarian tumor; epidermoid carcinoma, cell line; low-grade prostatic neoplasia; germ cell tumor; adrenal adenoma; normal prostate; colon tumor RER+; liver; pineal gland metastatic prostate bone lesion; epithelioid carcinoma.

It can be pointed out that levels of IL-8 decreased significantly in the cerebrospinal fluid (CSF) after cladribine treatment in relapsing-remitting multiple sclerosis (RR-MS; Bartosik-Psujek H et al. Acta Neurol Scand. 2004 June; 109(6):390-2. “Interleukin-8 and RANTES levels in patients with relapsing-remitting multiple sclerosis (RR-MS) treated with cladribine.”)

In addition, Kveine et al. have shown that TMEM9 mRNA is abundant in adrenal gland, thyroid gland, ovary, testis, and prostate, and to a lesser degree expressed in trachea, spinal cord, stomach, colon, small intestine, thymus, and spleen (Kveine et al. Biochem Biophys Res Commun. 2002 October. 4; 297(4):912-7. “Characterization of the novel human transmembrane protein 9 (JMEM9) that localizes to lysosomes and late endosomes.”). They also observed low expression in bone marrow, lymph node, and peripheral blood lymphocytes (PBL) and demonstrate that TMEM9 mRNA is expressed in hematopoietic cell lines of B lineage cell origin, T lineage cell origin, myeloid origin and erythroid origin.

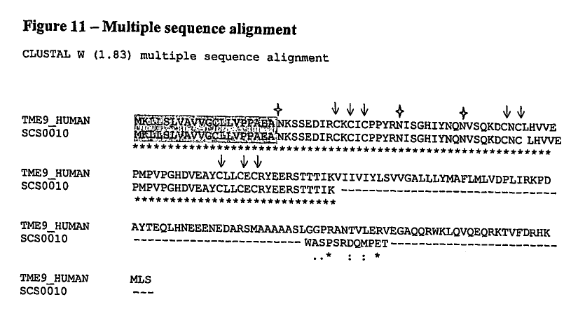

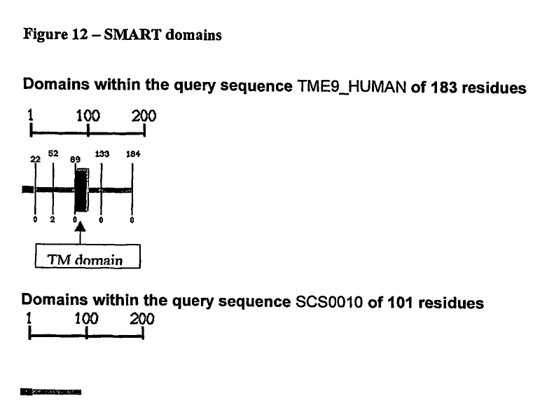

As such SCS0010 nucleic acid molecule, polypeptide, agonists or antagonists (e.g. agonistic or antagonistic antibodies) thereof may be useful in diagnosing or treating vascular diseases, inflammation, fibrosis, fibrotic disorders, rheumatoid arthritis, Crohn's disease, scleroderma, respiratory infections, asthma, allergic reactions, bronchitis, interstitial lung diseases, diabetic nephropathy, cardiac arrhythmia, cardiac hypertrophy, tumors, ulcers, ophthalmopathy, lipodystrophy, systemic sclerosis, osteoarthropathy, neuropathologies, pulmonary hypertension, hypoxia, hypertrophy/hyperplasia of pulmonary vascular smooth muscle, melanoma, leukemia and ovarian cancer, hepatocarcinogenesis, premalignant skin lesions (actinic keratosis), embryonic carcinoma, breast cancer, brain ependymoma, brain medulloblastoma, breast carcinoma, ductal carcinoma (breast), colon primary tumor, gastic cancer, primary malignant skin melanoma, prostate carcinoma (advanced tumor), nervous tumor, serous papillary carcinoma, endometrial adenocarcinoma, glioblastoma, poorly differentiated adenocarcinoma with signet ring cell features, anaplastic oligodendroglioma, lymphoma, squamous cell carcinoma, neuroblastoma, papillary serious carcinoma, adenocarcinoma, sarcomas, including myxoid liposarcoma, solitary fibrous tumor, malignant fibrous histiocytoma, gastrointestinal stromal tumor, mesothelioma, moderately differentiated adenocarcinoma, 2 pooled Wilms' tumors (primary and metastatic to brain), medulloblastoma, squamous cell carcinomas, Chondrosarcoma, carcinoma, retinoblastoma, renal cell adenocarcinoma, germ cell tumors, squamous cell carcinoma, cervical carcinoma, large cell carcinoma, small cell carcinoma, endometrium adenocarcinoma, melanotic melanoma, kidney tumor, choriocarcinoma, nervous tumor, hypemephroma, glioblastoma, acute myelogenous leukemia, anaplastic oligodendroglioma, adrenal cortex carcinoma, duodenal adenocarcinoma, hypemephroma, mucoepidermoid carcinoma, multiple sclerosis, epithelioid carcinoma, transitional cell papilloma, bladder tumor, parathyroid tumor, malignant melanoma, chronic lymphotic leukemia, primary lung cystic fibrosis, metastatic chondrosarcoma, leiomyosarcoma, chondrosarcoma Grade II, Lung Focal Fibrosis, hepatocellular carcinoma, papillary serous carcinoma, lung carcinoma, myeloma, teratocarcinoma, insulinoma, Enchondroma, epididymis, ovarian tumor, epidermoid carcinoma, cell line, prostatic neoplasia, germ cell tumor, adrenal adenoma, colon tumor, metastatic prostate bone lesion, epithelioid carcinoma as well as being useful in aging, tissue repair and plastic surgery. In addition, SCS0010 nucleic acid molecule, polypeptide, agonists or antagonists (e.g. agonistic or antagonistic antibodies) thereof may be useful in diagnosing or treating diseases (e.g. tumors) derived from or present in the organs and tissues were TMEM9 has been detected. Antagonists or agonists of SCS0010 (e.g. antibodies) can be directed to the sites and domains of SCS0010 as determined in example 5 and/or FIGS. 11 and 12. For example antibodies could be directed to at least one of the three conserved cystein-rich domains of SCS0010 (also present in integral TMEM9), which, without wishing to be bound to theory, might block dimerization of SCS0010 with itself or to other TMEM9 family members (e.g. integral TMEM9).

The therapeutic applications of the polypeptides of the invention and of the related reagents can be evaluated (in terms or safety, pharmacokinetics and efficacy) by the means of the in viVo/in vitro assays making use of animal cell, tissues and or by the means of in silico/computational approaches (Johnson D E and Wolfgang G H, 2000), known for the validation of IL-8-like polypeptides and other biological products during drug discovery and preclinical development.

The invention will now be described with reference to the specific embodiments by means of the following Examples, which should not be construed as in any way limiting the present invention. The content of the description comprises all modifications and substitutions which can be practiced by a person skilled in the art in light of the above teachings and, therefore, without extending beyond the meaning and purpose of the claims.

TABLE I

| Amino | | More Preferred |
|---|---|---|
| Acid | Synonymous Groups | Synonymous Groups |
| Ser | Gly, Ala, Ser, Thr, Pro | Thr, Ser |
| Arg | Asn, Lys, Gln, Arg, His | Arg, Lys, His |
| Leu | Phe, Ile, Val, Leu, Met | Ile, Val, Leu, Met |
| Pro | Gly, Ala, Ser, Thr, Pro | Pro |
| Thr | Gly, Ala, Ser, Thr, Pro | Thr, Ser |
| Ala | Gly, Thr, Pro, Ala, Ser | Gly, Ala |
| Val | Met, Phe, Ile, Leu, Val | Met, Ile, Val, Leu |
| Gly | Ala, Thr, Pro, Ser, Gly | Gly, Ala |
| Ile | Phe, Ile, Val, Leu, Met | Ile, Val, Leu, Met |
| Phe | Trp, Phe, Tyr | Tyr, Phe |
| Tyr | Trp, Phe, Tyr | Phe, Tyr |
| Cys | Ser, Thr, Cys | Cys |
| His | Asn, Lys, Gln, Arg, His | Arg, Lys, His |
| Gln | Glu, Asn, Asp, Gln | Asn, Gln |
| Asn | Glu, Asn, Asp, Gln | Asn, Gln |
| Lys | Asn, Lys, Gln, Arg, His | Arg, Lys, His |
| Asp | Glu, Asn, Asp, Gln | Asp, Glu |
| Glu | Glu, Asn, Asp, Gln | Asp, Glu |
| Met | Phe, Ile, Val, Leu, Met | Ile, Val, Leu, Met |
| Trp | Trp, Phe, Tyr | Trp |

TABLE II

| Amino | |
|---|---|
| Acid | Synonymous Groups |
| Ser | D-Ser, Thr, D-Thr, allo-Thr, Met, D-Met, Met(O), D-Met(O), L- |
| | Cys, D-Cys |
| Arg | D-Arg, Lys, D-Lys, homo-Arg, D-homo-Arg, Met, Ile, D-.Met, |
| | D-Ile, Orn, D-Orn |
| Leu | D-Leu, Val, D-Val, AdaA, AdaG, Leu, D-Leu, Met, D-Met |
| Pro | D-Pro, L-I-thioazolidine-4-carboxylic acid, D-or |
| | L-1-oxazolidine-4-carboxylic acid |
| Thr | D-Thr, Ser, D-Ser, allo-Thr, Met, D-Met, Met(O), D-Met(O), |
| | Val, D-Val |
| Ala | D-Ala, Gly, Aib, B-Ala, Acp, L-Cys, D-Cys |
| Val | D-Val, Leu, D-Leu, Ile, D-Ile, Met, D-Met, AdaA, AdaG |
| Gly | Ala, D-Ala, Pro, D-Pro, Aib, .beta.-Ala, Acp |
| Ile | D-Ile, Val, D-Val, AdaA, AdaG, Leu, D-Leu, Met, D-Met |
| Phe | D-Phe, Tyr, D-Thr, L-Dopa, His, D-His, Trp, D-Trp, |
| | Trans-3,4, or 5-phenylproline, AdaA, AdaG, |
| | cis-3,4, or 5-phenylproline, Bpa, D-Bpa |
| Tyr | D-Tyr, Phe, D-Phe, L-Dopa, His, D-His |
| Cys | D-Cys, S-Me-Cys, Met, D-Met, Thr, D-Thr |
| Gln | D-Gln, Asn, D-Asn, Glu, D-Glu, Asp, D-Asp |
| Asn | D-Asn, Asp, D-Asp, Glu, D-Glu, Gln, D-Gln |
| Lys | D-Lys, Arg, D-Arg, homo-Arg, D-homo-Arg, Met, |
| | D-Met, Ile, D-Ile, Orn, D-Orn |
| Asp | D-Asp, D-Asn, Asn, Glu, D-Glu, Gln, D-Gln |
| Glu | D-Glu, D-Asp, Asp, Asn, D-Asn, Gln, D-Gln |
| Met | D-Met, S-Me-Cys, Ile, D-Ile, Leu, D-Leu, Val, D-Val |

### EXAMPLES

### Example 1

Sequences of IL-8-like protein domains from the ASTRAL database (Brenner S E et al. “The ASTRAL compendium for protein structure and sequence analysis” Nucleic Acids Res. 2000 January 1; 28 (1): 254-6) were used to search for homologous protein sequences in genes predicted from human genome sequence (Celera version).

Those domain sequences were used to generate sequence profiles that include homologs. Additionally each profile position corresponding to a conserved cysteine that is involved in a disulfide bond were modified to require a strict AA conservation at those position. The sequence profiles of the IL-8-like domains were generated using PIMAII (Profile Induced Multiple Alignment; Boston University software, version II, Das S and Smith T F 2000). PIMAII algorithm iteratively aligns homologous sequences and generates a sequence profile.

The query protein sequences were obtained from the gene predictions and translations thereof (CELERA version of the human genome R27) as generated by one of three programs: the Genescan (Burge C, Karlin S., “Prediction of complete gene structures in human genomic DNA, J Mol. Biol. 1997 April 25; 268(1):78-94) Grail (Xu Y, Uberbacher E C., “Automated gene identification in large-scale genomic sequences”, J. Comput. Biol. 1997 Fall; 4(3):325-38) and Fgenesh (Proprietary Celera software).

The homology was detected using PIMAII that generates global-local alignments between a profile and a query sequence. In this case the algorithm was used with the profile of the IL-8-like functional domain as a query against a database of predicted human proteins. PIMAII compares the query profile to the database of gene predictions translated into protein sequence and can therefore identify a match to a DNA sequence that contains that domain.

Further comparison by BLAST (Basic Local Alignment Search Tool; NCBI version 2) of the sequence with known IL-8-like containing proteins identified the closest homolog (Gish W, States D J. “Identification of protein coding regions by database similarity search”, Nat. Genet. 1993 March; 3(3):266-72; Pearson W R, Miller W., “Dynamic programming algorithms for biological sequence comparison.”, Methods Enzymol. 1992; 210:575-601; Altschul S F et al., “Basic local alignment search tool”, J. Mol. Biol. 1990 Oct. 5; 215(3):403-10).

PIMAII parameters used for the detection were the PIMA prior amino acids probability matrix and a Z-cutoff score of 10. BLAST parameters used were: Comparison matrix=BLOSUM62; word length=3; .E value cutoff=10; Gap opening and extension=default; No filter.

Once the functional domain was identified in the sequence, the genes were re-predicted with the genewise algorithm using the sequence of the closets homolog. Birney E et al., “PairWise and SearchWise: finding the optimal alignment in a simultaneous comparison of a protein profile against all DNA translation frames”, Nucleic Acids Res. 1996 Jul. 15; 24(14):2730-9)

The profiles for homologous domains IL-8-like were generated automatically using the PSI-BLAST (Altshul et al. 1997), scripts written in PERL (Practical Extraction and Report Language) and PIMAIL.

A total of 51 (IL-8-like) 18 predicted genes were initially selected since they were judged as potentially novel.

The novelty of the protein sequences was assessed by searching protein databases (SwissProt/Trembl, Human IPI and Derwent GENESEQ) using BLAST. The match to a functional domain and the novelty were checked by a “manual” inspection for the selected novel candidates.

### Example 2

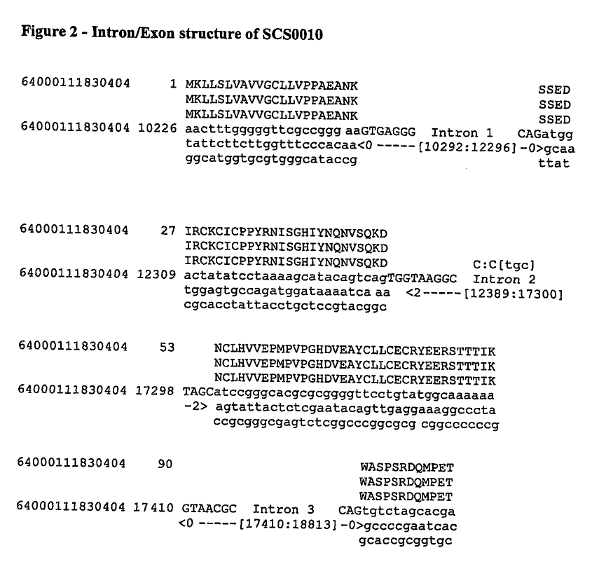

One sequence isolated by the methodology set out in Example 1 is that referred to herein as SCS0010 . The most similar known polypeptide sequence is a Human NF-kB activating protein, with accession number ABP61498, however the third exon is different. FIG. 2 shows the intron/exon structure of the gene that encodes SCS0010 (64000111830404).

### Example 3

### Identification and Cloning of Splice Variants of SCS0010

1. Cloning of SCS0010

1.1 cDNA Libraries

A human placenta cDNA library (in bacteriophage lambda (λ) GT10) was purchased from Clontech (cat. no. HL1075b). Bacteriophage λ DNA was prepared from a small scale cultures of infected E. coli host strain using the Wizard Lambda Preps DNA purification system according to the manufacturer's instructions (Promega, Corporation, Madison Wis.). The resultant phage DNA was resuspended at a concentration of 50 ng/μl and used in subsequent PCR reactions.

1.2 Gene Specific Cloning Primers for PCR

A pair of PCR primers having a length of between 18 and 25 bases were designed for amplifying the coding sequence of the virtual cDNA using Primer Designer Software (Scientific & Educational Software, PO Box 72045, Durham, N. C. 27722-2045, USA). PCR primers were optimized to have a Tm close to 55±10° C. and a GC content of 40-60%. Primers were selected which had high selectivity for the target sequence (little or no none specific priming).

1.3 PCR of SCS0010 cDNA from Human Placenta Phase cDNA Library

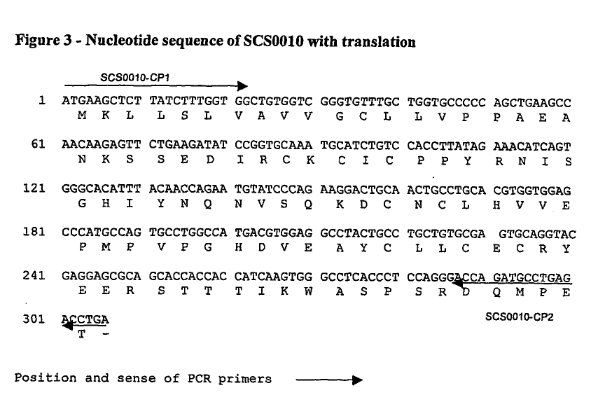

Gene-specific PCR amplification primers (SCS0010 -CP1 and SCS0010-CP2, FIG. 3 and Table 4) were designed to amplify a 299 bp product expected to contain almost the predicted coding sequence of SCS0010. The PCR was performed in a final volume of 50 μl containing 1× AmpliTaq™ buffer, 200 μM dNTPs, 50 pmoles each of cloning primers, 2.5 units of AmpliTaq™ (Perkin Elmer) and 100 ng of each phage library pool DNA using an MT Research DNA Engine, programmed as follows: 94° C., 1 min; 40 cycles of 94° C., 1 min, x° C., and 1 min at 72° C.; followed by 1 cycle at 72° C. for 7 min and a holding cycle at 4° C.

The amplification products were visualized on 0.8% agarose gels in 1× TAE buffer (Invitrogen) and were purified from the gel using the Wizard PCR Preps DNA Purification System (Promega). PCR products eluted in 50 l of sterile water were either subcloned directly or stored at −20° C.

1.4 Subcloning of PCR Products

PCR products were subcloned into the topoisomerase I modified cloning vector (pCR4-TOPO) using the TOPO cloning kit purchased from the Invitrogen Corporation using the conditions specified by the manufacturer. Briefly, 4 μl of gel purified PCR product was incubated for 15 min at room temperature with 1 μl of TOPO vector and 1 μl salt solution. The reaction mixture was then transformed into E. coli strain TOP10 (Invitrogen) as follows: a 50 μaliquot of One Shot TOP10 cells was thawed on ice and 2 μl of TOPO reaction was added. The mixture was incubated for 15 min on ice and then heat shocked by incubation at 42° C. for exactly 30 s. Samples were returned to ice and 250 μl of warm SOC media (room temperature) was added. Samples were incubated with shaking (220 rpm) for 1 h at 37° C. The transformation mixture was then plated on L-broth (LB) plates containing ampicillin (100 μg/ml) and incubated overnight at 37° C. Ampicillin resistant colonies containing inserts were identified by colony PCR.

1.4 Colony PCR

Colonies were inoculated into 50 μl sterile water using a sterile toothpick. A 10 μl aliquot of the inoculum was then subjected to PCR in a total reaction volume of 20 μl as described above, except the primers used were T7 and T3. The cycling conditions were as follows: 94° C., 2 min; 30 cycles of 94° C., 30 sec, 48° C., 30 sec and 72° C., 1 min. Samples were then maintained at 4° C. (holding cycle) before further analysis.

PCR reaction products were analyzed on 1% agarose gel in 1×TAE buffer. Colonies which gave the expected PCR product size (approximately 299 bp cDNA+105 bp due to the multiple cloning site or MCS) were grown up overnight at 37° C. in 5 ml L-Broth (LB) containing ampicillin (100 μg/ml), with shaking (220 rpm).

1.5 Plasmid DNA Preparation and Sequencing

Miniprep plasmid DNA was prepared from 5 ml cultures using a Qiaprep Turbo 9600 robotic system (Qiagen) or Wizard Plus SV Minipreps kit (Promega cat. no. 1460) according to the manufacturer's instructions. Plasmid DNA was eluted in 100 μl of sterile water. The DNA concentration was measured using an Eppendorf BO photometer. Plasmid DNA (200-500 ng) was subjected to DNA sequencing with T7 and T3 primers using the BigDyeTerminator system (Applied Biosystems cat. no. 4390246) according to the manufacturer's instructions. The primer sequences are shown in Table 3. Sequencing reactions were purified using Dye-Ex columns (Qiagen) or Montage SEQ 96 cleanup plates (Millipore cat. no. LSKS09624) then analyzed on an Applied Biosystems 3700 sequencer.

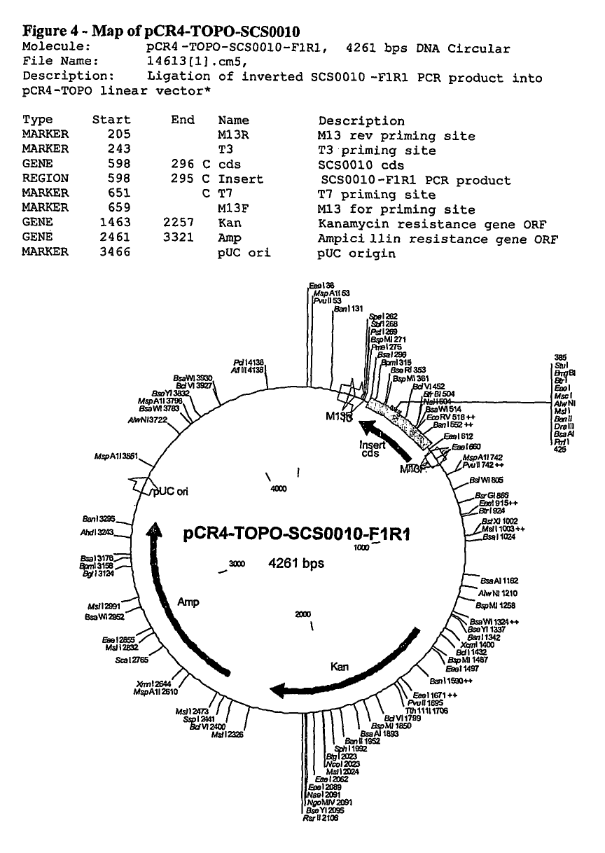

Sequence analysis identified one clone which contained 100% match to the predicted SCS0010 sequence (FIG. 3). The plasmid map of the cloned PCR product (pCR4-TOPO-SCS0010) (plasmid ID.14613) is shown in FIG. 4.

2. Construction of Mammalian Cell Expression Vectors for SCS0010

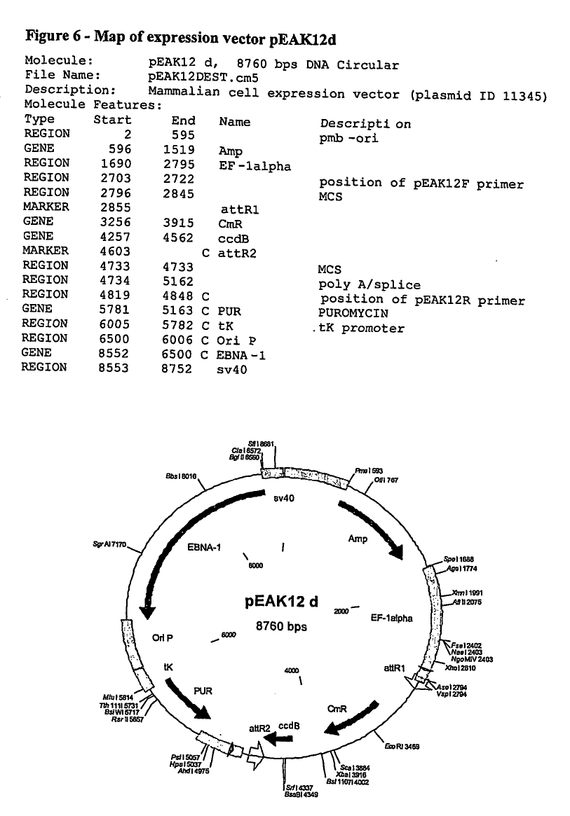

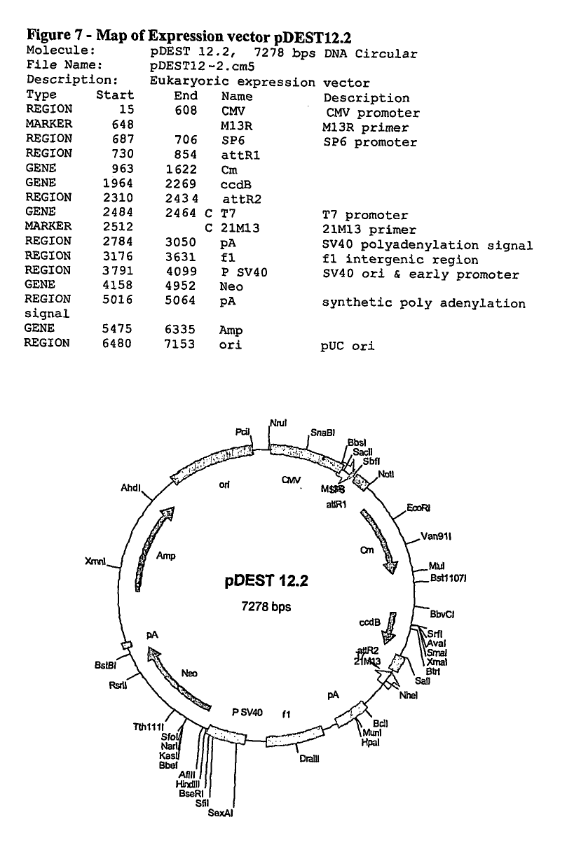

A pCR4-TOPO clone containing the coding sequence (ORF) of SCS0010 identified by DNA sequencing (pCR4-TOPO-SCS0010, plasmid ID. 14613) (FIG. 4) was then used to subclone the insert into the mammalian cell expression vectors pEAK12d (FIG. 6) and pDEST12.2 (FIG. 7) using the Gateway™ cloning methodology (Invitrogen).

2.1 Generation of Gateway Compatible SCS0010 ORF Fused to an In Frame 6HIS Tag Sequence.

The first stage of the Gateway cloning process involves a two step PCR reaction which generates the ORF of SCS0010 flanked at the 5′ end by an attB1 recombination site and Kozak sequence, and flanked at the 3′ end by a sequence encoding an in frame 6 histidine (6HIS) tag, a stop codon and the attB2 recombination site (Gateway compatible cDNA). The first PCR reaction (in a final volume of 50 μl) contains: 1 μl (40 ng) of pCR4-TOPO-SCS0010 (plasmid ID 14613), 1.5 μl dNTPs (10 mM), 10 μl of 10× Pfx polymerase buffer, 1 μl MgSO4 (50 mM), 0.5 μl each of gene specific primer (100 μM) (SCS0010-EX1 and SCS0010-EX2), 2.5 μl 10×Enhancer™ solution (Invitrogen) and 0.5 μl Platinum Pfx DNA polymerase (Invitrogen). The PCR reaction was performed using an initial denaturing step of 95° C. for 2 min, followed by 12 cycles of 94° C. for 15 s; 55° C. for 30 s and 68° C. for 2 min; and a holding cycle of 4° C. The amplification products were visualized on 0.8% agarose gel in 1×TAE buffer (Invitrogen) and a product migrating at the predicted molecular mass was purified from the gel using the Wizard PCR Preps DNA Purification System (Promega) and recovered in 50 μl sterile water according to the manufacturer's instructions.

The second PCR reaction (in a final volume of 50 μl) contained 10 μl purified PCR 1 product, 1.5 μl dNTPs (10 mM), 5 μl of 10× Pfx polymerase buffer, 1 μl MgSO4 (50 mM), 0.5 μl of each Gateway conversion primer (100 μM) (GCP forward and GCP reverse) and 0.5 μl of Platinum Pfx DNA polymerase. The conditions for the 2nd PCR reaction were: 95° C. for 1 min; 4 cycles of 94° C., 15 sec; 50° C., 30 sec and 68° C. for 2 min; 25 cycles of 94° C., 15 sec; 55° C., 30 sec and 68° C., 2 min; followed by a holding cycle of 4° C. PCR products were gel purified using the Wizard PCR prep DNA purification system (Promega) according to the manufacturer's instructions.

2.2 Subcloning of Gateway Compatible SCS0010 ORF into Gateway Entry Vector pDONR221 and Expression Vectors pEAK12d and pDEST12.2

The second stage of the Gateway cloning process involves subcloning of the Gateway modified PCR product into the Gateway entry vector pDONR221 (Invitrogen, FIG. 3) as follows: 5 μl of purified product from PCR2 were incubated with 1.5 μl pDONR221 vector (0.1 μg/μl), 2 μl BP buffer and 1.5 μl of BP clonase enzyme mix (Invitrogen) in a final volume of 10 μl at RT for 1 h. The reaction was stopped by addition of proteinase K 1 μl (2 μg/μl) and incubated at 37° C. for a further 10 min. An aliquot of this reaction (1 μl) was used to transform E. coli DH10B cells by electroporation as follows: a 25 μl aliquot of DH10B electrocompetent cells (Invitrogen) was thawed on ice and 1 μl of the BP reaction mix was added. The mixture was transferred to a chilled 0.1 cm electroporation cuvette and the cells electroporated using a BioRad Gene-Pulser™ according to the manufacturer's recommended protocol. SOC media (0.5 ml) which had been pre-warmed to room temperature was added immediately after electroporation. The mixture was transferred to a 15 ml snap-cap tube and incubated, with shaking (220 rpm) for 1 h at 37° C. Aliquots of the transformation mixture (10 μl and 50 μl) were then plated on L-broth (LB) plates containing kanamycin (40 μg/ml) and incubated overnight at 37° C.

Plasmid mini-prep DNA was prepared from 5 ml cultures from 6 of the resultant colonies using a Qiaprep Turbo 9600 robotic system (Qiagen). Plasmid DNA (150-200 ng) was subjected to DNA sequencing with 21M13 and M13Rev primers using the BigDyeTerminator system (Applied Biosystems cat. no. 4390246) according to the manufacturer's instructions. The primer sequences are shown in Table 3. Sequencing reactions were purified using Dye-Ex columns (Qiagen) or Montage SEQ 96 cleanup plates (Millipore cat no. LSKS09624) then analyzed on an Applied Biosystems 3700 sequencer.

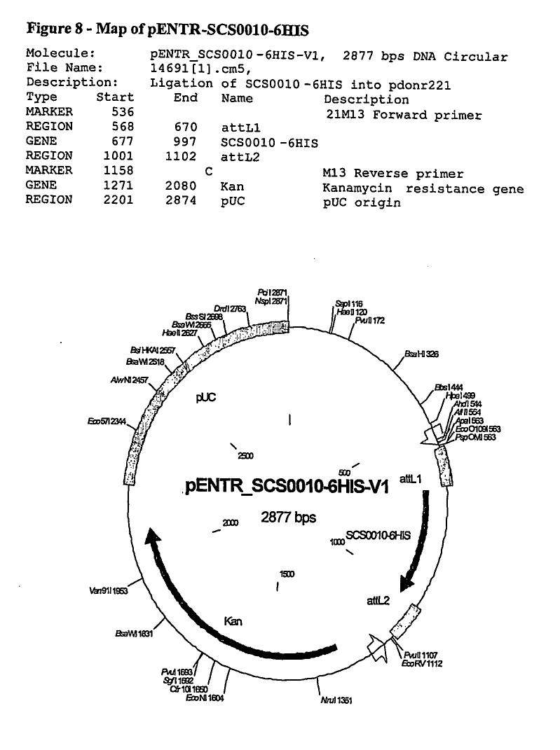

Plasmid eluate (2 μl or approx. 150 ng) from one of the clones which contained the correct sequence (pENTR-SCS0010-6HIS, plasmid ID 14691, FIG. 8) was then used in a recombination reaction containing 1.5 μl of either pEAK12d vector or pDEST12.2 vector (FIGS. 4 & 5) (0.1 μg/μl), 2 μl LR buffer and 1.5 μl of LR clonase (Invitrogen) in a final volume of 10 μl. The mixture was incubated at RT for 1 h, stopped by addition of proteinase K (2 μg) and incubated at 37° C. for a further 10 min. An aliquot of this reaction (1 μl) was used to transform E. coli DH10B cells by electroporation as follows: a 25 μl aliquot of DH10B electrocompetent cells (Invitrogen) was thawed on ice and 1 μl of the LR reaction mix was added. The mixture was transferred to a chilled 0.1 cm electroporation cuvette and the cells electroporated using a BioRad Gene-Pulser™ according to the manufacturer's recommended protocol. SOC media (0.5 ml) which had been pre-warmed to room temperature was added immediately after electroporation. The mixture was transferred to a 15 ml snap-cap tube and incubated, with shaking (220 rpm) for 1 h at 37° C. Aliquots of the transformation mixture (10 μl and 50 μl) were then plated on L-broth (LB) plates containing ampicillin (100 μg/ml) and incubated overnight at 37° C.

Plasmid mini-prep DNA was prepared from 5 ml cultures from 6 of the resultant colonies subcloned in each vector using a Qiaprep Turbo 9600 robotic system (Qiagen). Plasmid DNA (200-500 ng) in the pEAK12d vector was subjected to DNA sequencing with pEAK12F and pEAK12R primers as described above. Plasmid DNA (200-500 ng) in the pDEST12.2 vector was subjected to DNA sequencing with 21M13 and M13Rev primers as described above. Primers sequences are shown in Table 3.

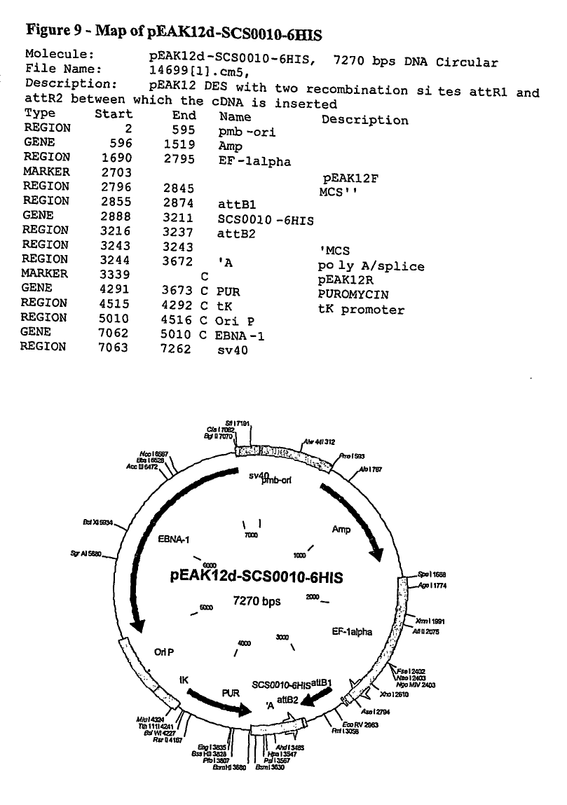

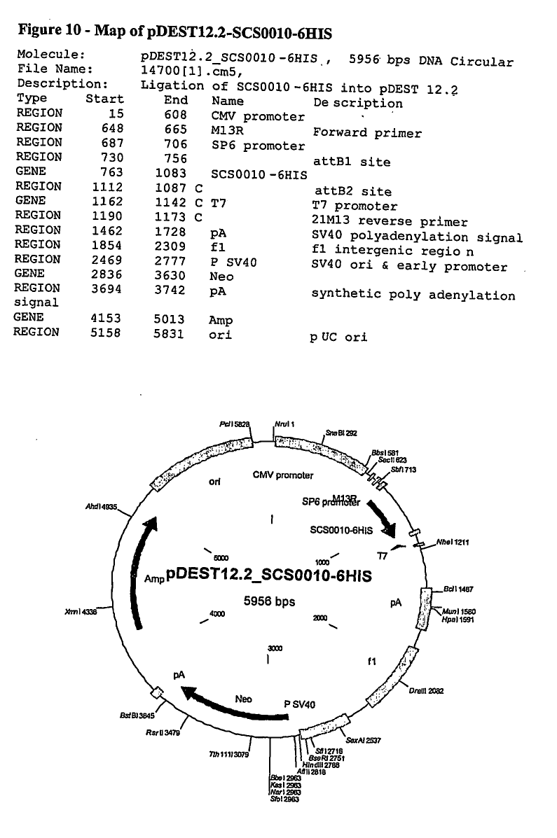

CsCl gradient purified maxi-prep DNA was prepared from a 500 ml culture of one of each of the sequence verified clones (pEAK12d-SCS0010-6HIS, plasmid ID number 14699, FIG. 9, and pDEST12.2-SCS0010-6HIS, plasmid ID 14700, FIG. 10) using the method described by Sambrook J. et al., 1989 (in Molecular Cloning, a Laboratory Manual, 2^{nd} edition, Cold Spring Harbor Laboratory Press), Plasmid DNA was resuspended at a concentration of 1 μg/μl in sterile water (or 10 mM Tris-HCl pH 8.5) and stored at −20° C.

TABLE 3
SCS0010 cloning and sequencing primers

| | |
|---|---|
| Primer | Sequence (5′-3′) |

| | |
|---|---|
| SCS0010-CP1 | ATG AAG CTC TTA TCT TTG GTG G |
| SCS0010-CP2 | AGG TCT CAG GCA TCT GGT C |
| SCS0010-EX1 | AA GCA GGC TTC GCC ACC ATG AAG CTC TTA |
| | TGT TTG GT |
| SCS0010-EX2 | GTG ATG GTG ATG GTG GGT CTC AGG CAT CTG |
| | GTC CC |
| GCP Forward | G GGG ACA AGT TTG TAC AAA AAA GCA GGC |
| | TTC GCC ACC |
| GCP Reverse | GGG GAC CAC TTT GTA CAA GAA AGC TGG |
| | GTT TCA ATG GTG ATG GTG ATG GTG |
| pEAK12F | GCC AGC TTG GCA CTT GAT GT |
| pEAK12R | GAT GGA GGT GGA GGT GTC AG |
| 21M13 | TGT AAA ACG ACG GCC AGT |
| M13REV | CAG GAA ACA GCT ATG ACC |
| T7 | TAA TAC GAC TCA CTA TAG G |
| T3 | ATT AAC CCT CAC TAA AGG |

Underlined sequence = Kozak sequence
Bold = Stop codon
Italic sequence = His tag

### Example 4Expression and Purification of SCS0010

1. Functional Genomics Throughput Expression in Mammalian Cells and Purification of the Cloned, His-Tagged Plasmid

Human Embryonic Kidney 293 cells expressing the Epstein-Barr virus Nuclear Antigen (HEK293-EBNA, Invitrogen) were maintained in suspension in Ex-cell VPRO serum-free medium (seed stock, maintenance medium, JRH). On the day of transfection, cells were counted, centrifuged (low speed) and the pellet re-suspended into the desired volume of FEME medium (see below) supplemented with 1% FCS to yield a cell concentration of 1XE6 viable cells/ml. The cDNA was diluted at 2 mg/liter volume (co-transfected with 2% eGFP) in FEME (200 ml/litre volume). PolyEthyleneImine transfection agent (4 mg/litre volume) was then added to the cDNA solution, vortexed and incubated at room temperature for 10 minutes (generating the transfection Mix).

This transfection mix was then added to the spinner and incubated for 90 minutes in a CO₂ incubator (5% CO₂ and 37° C.). Fresh FEME medium (1% FCS) was added after 90 minutes such as to double the initial spinner volume. The spinner was then incubated for 6 days. On day 6 (harvest day), spinner supernatant (500 ml) was centrifuged (4° C., 400 g) and placed into a pot bearing a unique identifier with plasmid number and fermentation number.

Purification Process

The 500 ml culture medium sample containing the recombinant protein with a C-terminal 6His tag were diluted with one volume cold buffer A (50 mM NaH₂PO₄; 600 mM NaCl; 8.7% (w/v) glycerol, pH 7.5) to a final volume of 1000 ml. The sample was filtered through a 0.22 μm sterile filter (Millipore, 500 ml filter unit) and kept at 4° C. in a 1 liter sterile square media bottle (Nalgene).

The purification was performed at 4° C. on the VISION workstation (Applied Biosystems) connected to an automatic sample loader (Labomatic). The purification procedure was composed of two sequential steps, metal affinity chromatography on a Poros 20 MC (Applied Biosystems) column charged with Ni ions (10×50 mm, 3.93 ml), followed by buffer exchange on a Sephadex G-25 medium (Amersham Pharmacia) gel filtration column (1,0×15 cm).

For the first chromatography step the metal affinity column was regenerated with 30 column volumes of EDTA solution (100 mM EDTA; 1 M NaCl; pH 8.0), recharged with Ni ions through washing with 15 column volumes of a 100 mM NiSO₄ solution, washed with 10 column volumes of buffer A, followed by 7 column volumes of buffer B (50 mM NaH₂PO₄; 600 mM NaCl; 8.7% (w/v) glycerol, 400 mM; imidazole, pH 7.5), and finally equilibrated with 15 column volumes of buffer A containing 15 mM imidazole. The sample was transferred, by the Labomatic sample loader, into a 200 ml sample loop and subsequently charged onto the Ni metal affinty column at a flow rate of 20 ml/min. The charging procedure was repeated 5 times in order to transfer the entire sample (1000 ml) onto the Ni column. Subsequently the column was washed with 12 column volumes of buffer A, followed by 28 column volumes of buffer A containing 20 mM imidazole. During the 20 mM imidazole wash, loosely attached contaminating proteins were eluted of the column. The recombinant His-tagged protein was finally eluted with 10 column volumes of buffer B at a flow rate of 2 ml/min, and the eluted protein was collected in a 2.7 ml fraction.

For the second chromatography step, the Sephadex G-25 gel-filtration column was regenerated with 2 ml of buffer D (1.137 M NaCl; 2.7 mM KCl; 1.5 mM KH₂PO₄; 8 mM Na₂HPO₄; pH 7.2), and subsequently equilibrated with 4 column volumes of buffer C (137 mM NaCl; 2.7 mM KCl; 1.5 mM KH₂PO₄; 8 mM Na₂HPO₄; 20% (w/v) glycerol; pH 7.4). The peak fraction eluted from the Ni-column was automatically, through the integrated sample loader on the VISION, loaded onto the Sephadex G-25 column and the protein was eluted with buffer C at a flow rate of 2 ml/min. The desalted sample was recovered in a 2.7 ml fraction. The fraction was filtered through a 0.22 μm sterile centrifugation filter (Millipore), aliquoted, frozen and stored at −80° C. An aliquot of the sample was analyzed on SDS-PAGE (4-12% NuPAGE gel; Novex) by coomassie staining and Western blot with anti-His antibodies.

Coomassie staining. The NuPAGE gel was stained in a 0.1% coomassie blue R250 staining solution (30% methanol, 10% acetic acid) at room temperature for 1 h and subsequently distained in 20% methanol, 7.5% acetic acid until the background was clear and the protein bands clearly visible.

Western Blot

Following the electrophoresis the proteins were electrotransferred from the gel to a nitrocellulose membrane at 290 mA for 1 hour at 4° C. The membrane was blocked with 5% milk powder in buffer E (137 mM NaCl; 2.7 mM KCl; 1.5 mM KH₂PO₄; 8 mM Na₂HPO4; 0.1% Tween 20, pH 7.4) for 1 h at room temperature, and subsequently incubated with a mixture of 2 rabbit polyclonal anti-His antibodies (G-18 and H-15, 0.2 ug/ml each; Santa Cruz) in 2.5% milk powder in buffer E overnight at 4° C. After further 1 hour incubation at room temperature, the membrane was washed with buffer E (3×10 min), and then incubated with a secondary HRP-conjugated anti-rabbit antibody (DAKO, HRP 0399) diluted 1/3000 in buffer B containing 2.5% milk powder for 2 hours at room temperature. After washing with buffer E (3×10 minutes), the membrane was developed with the ECL kit (Amersham Pharmacia) for 1 min. The membrane was subsequently exposed to a Hyperfilm (Amersham Pharmacia), the film developed and the western blot image visually analyzed.

Protein Assay

The protein concentration was determined using the BCA protein assay kit (Pierce) with bovine serum albumin as standard. The recovery was 420 μg.

### Example 5

### Characterization of SCS0010

Using Blast, SCS0010 was determined to be a splice variant of transmembrane protein 9 (TMEM9), also known as Dermal papilla derived protein 4 (DERP4), HSPC186 or UNQ631/PRO1248 (SwissProt entry Q9POT7, TME9_HUMAN). The alignment between TME9_HUMAN and SCS0010 is shown in FIG. 11, indicating that SCS0010 differs at the 3′ end. TMEM9 known features are:
183 amino acids long (20574 Da),
type I membrane protein, localized in late endosomes and lysosomes,
three glycosylated forms,
possible dimerization,
belongs to the TMEM9 family and
suggested role in intracellular transport.

TMEM9 has been characterized by Kveine et al. (Kveine et al. Biochem Biophys Res Commun. 2002 Oct. 4; 297(4):912-7. “Characterization of the novel human transmembrane protein 9 (TMEM9) that localizes to lysosomes and late endosomes.”). The TMEM9 family is described as following in Interpro (http://www.ebi.ac.uk/interpro: “This family contains several eukaryotic transmembrane proteins which are homologous to Homo sapiens transmembrane protein 9 SWISSPROT:09POT7. The TMEM9 gene encodes a 183 amino-acid protein that contains an N-terminal signal peptide, a single transmembrane region, three potential N-glycosylation sites and three conserved cys-rich domains in the N terminus, but no known functional domains. The protein is highly conserved between species from Caenorhabditis elegans to H. sapiens and belongs to a novel family of transmembrane proteins. The exact function of TMEM9 is unknown although it has been found to be widely expressed and localised to the late endosomes and lysosomes MEDLINE:. Members of this family contain CXCXC repeats INTERPRO:IPR004153 in their N-terminal region.”. Interleukin-8 (IL-8) is a member of CXC chemokine subfamily and SCS0010 displays common patterns to IL-8, including CXCX repeats. Bioinformatic tools, including SMART (http://smart.embl-heidelberg.de/), were used to identify the putative domains of TMEM9 and of the splice variant SCS0010. Results of SMART are shown in FIG. 12. This analysis indicates that SCS0010 is a soluble splice variant of TMEM9, thus indicating that SCS0010 display unique functionalities relative to the membrane bound TMEM9. As such, the secreted SCS0010 protein may act as an antagonist of the integral TMEM9 in vivo. Kveine et al. indicate that TMEM9 has three conserved cystein-rich domains that are found in the N-terminal regions. They further note that these regions may participate in protein folding, protein interactions, and multimerization. These regions are fully conserved in SCS0010 (indicated by arrows in FIG. 12). As such, and without wishing to be bound to theory, soluble SCS0010 might be involved in the regulation of TMEM9 by dimerization. Thus, SCS0010 might show particularly useful in the diagnosis and treatment of diseases, as described in the therapeutic uses section. Motifs and sites of SCS0010 herein identified (e.g. glycosylation sites, proteoglycan sites, SH2 domain, conserved cystein rich domains) can be modified or/and targeted (by agonists or antagonists, e.g. antibodies) for the purpose of treatment and/or diagnosis.

Without wishing to be bound to theory, mutations in the TMEM9 site for proteoglycans (see ELM results below) might be associated to an inherited predisposition to cancer. Three N-glycosylation sites in the N-terminal part of TNEM9 identified by Kveine et al. were suggested to be of potential importance for the protein structure and function. They have identified three glycosylated forms that may have distinct functional and/or affinity properties. The association of TMEM9 with cancer is also supported by a publication from Kurokawa et al. (Kurokawa et al. J Exp Clin Cancer Res. 2004 March; 23(1):135-41. PCR-array gene expression profiling of hepatocellular carcinoma.), as well as by microarray-based and other expression experiments (see therapeutic uses). Thus, soluble SCS0010, by binding to TMEM9, might be able to reduce and/or prevent a number of diseases including cancer.

Prosite (http://us.expasy.org/prosite/) and ELM (http://elm.eu.org/basicELM/) were also run on the sequence.

Prosite Results:

>PDOC00001 PS00001 ASN_GLYCOSYLATION N-glycosylation site [pattern] [Warning: pattern with a high probability of occurrence].
25-28 NKSS
42-45 NISG
51-54 NVSQ

>PDOC00005 PS00005 PKC_PHOSPHO_SITE Protein kinase C phosphorylation site [pattern] [Warning: pattern with a high probability of occurrence].
53-55 SqK
91-93 TiK

>PDOC00006 PS00006 CK2_PHOSPHO_SITE Casein kinase II phosphorylation site [pattern] [Warning: pattern with a high probability of occurrence].
27-30 SseD

53-56 SqkD

TABLE 4
ELM results:

| | Instances | | | | |
|---|---|---|---|---|---|
| | (Matched | | | Cell | |
| Elm Name | Sequence) | Positions | Elm Description | Compartment | Pattern |
| MOD GlcNHglycan | ISGH | 39-42 | Glycosaminoglycan | extracellular, | [ED]{0, 3}.(S) |
| | | | attachment site | Golgi | [GA]. |
| | | | | apparatus | |
| MOD N-GLC 1 | NIS | 38-40 | Generic motif for N- | extracellular, | (N)[\hat{ }P][ST]\|(N) |
| | NVS | 47-49 | glycosylation. Shakin- | Golgi | [\hat{ }P][ST][\hat{ }P] |
| | | | Eshleman et al. showed that | apparatus, | |
| | | | Trp, Asp, and Glu are | endoplasmic | |
| | | | uncommon before the | reticulum | |
| | | | Ser/Thr position. Efficient | | |
| | | | glycosylation usually occurs | | |
| | | | when ˜60 residues or more | | |
| | | | separate the glycosylation | | |
| | | | acceptor site from the C- | | |
| | | | terminus | | |
| LIG SH2 STAT5 | YCLL | 72-75 | STAT5 Src Homology 2 | not annotated | Y[VLTFIC] . . . |
| | | | (SH2) domain binding | | |
| | | | motif. | | |

Description of ELM Results (According to ELM)

MOD_GlcNHglycan: Proteoglycans are found at the cell surface and in the extracellular matrix. They are important for cell communication, playing a role for example in morphogenesis and development. Mutations in some proteoglycans are associated with an inherited predisposition to cancer. The core protein is modified by attachment of the glycosaminoglycan chain at an exposed serine residue. For heparan sulphate, the process begins by transfer of xylose from UDP-xylose to the serine hydroxyl group by protein xylosyl transferase (EC 2.4.2.26) in the Golgi stack. The system appears to have evolved in metazoan animals.

MOD_N-GLC_{—}1: N-glycosylation is the most common modification of secretory and membrane-bound proteins in eukaryotic cells. The whole process of N-glycosylation. comprises more than 100 enzymes and transport proteins. The biosynthesis of all N-linked oligosaccharides begins in the ER with a large precursor oligosaccharid. The structure of this oligosaccharide [(Glc)3(Man)9(GIcNAc)2] is the same in plants, animals, and single cell eukaryotes. This precursor is linked to a dolichol, a long-chain polyisoprenoid lipid that act as a carrier for the oligosaccharide. The oligosaccharide then is transfer by an ER enzyme from the dodichol carrier to an asparagine residue on a nascent protein. The oligosaccharide chain is then processed as the glycoprotein moves through the Golgi apparatus. In some cases this modification involves attachment of more mannose groups; in other cases a more complex type of structure is attached.

LIG_SH2_STAT5: Src Homology 2 (SH2) domains are small modular domains found within a great number of proteins involved in different signaling pathways. They are able to bind specific motifs containing a phopshorylated tyrosine residue, propagating the signal downstream promoting protein-protein interaction and/or modifying enzymatic activities. Different families of SH2 domains may have different binding specifity, which is usually determined by few residues C-terminal with respect to the pY (positions+1, +2 and +3. Non-phosphorylated peptides do not bind to the SH2 domains. At least three different binding motifs are known: pYEEI (Src-family SH2 domains), pY[IV].[VILP] (SH-PTP2, phospholipase C-gamma), pY.[EN] (GRB2). The interaction between SH2 domains and their substrates is however dependent also to cooperative contacts of other surface regions.

### Example 6

### Cell- and Animal-Based Assay for the Validation and Characterization of the Chemokine-Like Polypeptides

Studies on structure-activity relationships indicate that chemokines bind and activate receptors by making use of the amino-terminal region. Proteolytic digestion, mutagenesis, or chemical modifications directed to amino acids in this region can generate compounds having antagonistic activity (Loetscher P and Clark-Lewis I, J Leukoc Biol, 69: 881-884, 2001 Lambeir A, et al. J Biol Chem, 276: 29839-29845, 2001, Proost P, et al. Blood, 98 (13):3554-3561, 2001). Thus, antagonistic molecules resulting from specific modifications (deletions, non-conservative substitutions) of one or more residues in the amino-terminal region or in other regions of the corresponding chemokine are considered having therapeutic potential for inflammatory and autoimmune diseases (WO 02/28419; WO 00/27880; WO 99/33989; Schwarz MK and Wells T N, Curr Opin Chem Biol, 3: 407-17, 1999). Therefore, a further object of the present patent application is represented by such kind of antagonists generated by modifying the polypeptides of the invention.

The therapeutic applications of the polypeptides of the invention and of the related reagents can be evaluated (in terms or safety, pharmacokinetics and efficacy) by the means of the in vivo/in vitro assays making use of animal cell, tissues and models (Coleman R A et al., Drug Discov Today, 6: 1116-1126, 2001; Li A P, Drug Discov Today, 6: 357-366, 2001; Methods Mol. Biol. vol. 138, “Chemokines Protocols”, edited by Proudfoot A I et al., Humana Press Inc., 2000; Methods Enzymol, vol. 287 and 288, Academic Press, 1997), or by the means of in silico/computational approaches (Johnson D E and Wolfgang G H, Drug Discov Today, 5: 445-454, 2000), known for the validation of chemokines and other biological products during drug discovery and preclinical development.

The present patent application discloses novel chemokine-like polypeptides and a series of related reagents that may be useful, as active ingredients in pharmaceutical compositions appropriately formulated, in the treatment or prevention of diseases such as cell proliferative disorders, autoimmune/inflammatory disorders, cardiovascular disorders, neurological disorders, developmental disorders, metabolic disorder, infections and other pathological conditions. In particular, given the known properties of chemokines, the disclosed polypeptides and reagents should address conditions involving abnormal or defective cell migration. Non-limitative examples of such conditions are the following: arthritis, rheumatoid arthritis (RA), psoriatic arthritis, osteoarthritis, systemic lupus erythematosus (SLE), systemic sclerosis, scleroderma, polymyositis, glomerulonephritis, fibrosis, lung fibrosis and inflammation, allergic or hypersensitvity diseases, dermatitis, asthma, chronic obstructive pulmonary disease (COPD), inflammatory bowel disease (IBD), Crohn's diseases, ulcerative colitis, multiple sclerosis, septic shock, HIV infection, transplant rejection, wound healing, metastasis, endometriosis, hepatitis, liver fibrosis, cancer, analgesia, and vascular inflammation related to atherosclerosis.

Several assays have been developed for testing specificity, potency, and efficacy of chemokines using cell cultures or animal models, for example in vitro chemotaxis assays (Proudfoot A, et al. J Biol Chem 276: 10620-10626, 2001; Lusti-Narasimhan M et al., J Biol Chem, 270: 2716-21, 1995), or mouse ear swelling (Garrigue J L et al., Contact Dermatitis, 30: 231-7, 1994). Many other assays and technologies for generating useful tools and products (antibodies, transgenic animals, radiolabeled proteins, etc.) have been described in reviews and books dedicated to chemokines (Methods Mol. Biol vol. 138, “Chemokines Protocols”, edited by Proudfoot Al et al., Humana Press Inc., 2000; Methods Enzymol, vol. 287 and 288, Academic Press, 1997), and can be used to verify, in a more precise manner, the biological activities of the chemokine-like polypeptides of the invention and related reagents in connection with possible therapeutic or diagnostic methods and uses.

Cytokine Expression Modulation Assays

1 Introduction

The following in vitro cell-based tri-replicas assays measure the effects of the protein of the invention on cytokine secretion induced by Concanavalin A (Con A) acting on different human peripheral blood mononuclear cells (hPBMC) cells as measured by a cytokine bead array (CBA) assay for IL-2, IFNγ, TNF-α, IL-5, IL-4 and IL-10.

The optimal conditions are 100000 cells/well in 96-well plates and 100 μl final in 2% glycerol.

The optimal concentration of mitogen (ConA) is 5 ng/ml.

The optimal time for the assay is 48 h.

The read-out choice is the CBA.

2 Equipments and Softwares
96 well microtiter plate photometer EX (Labsystem).
Graph Pad Prism Software
Excel software
Flow cytometer Becton-Dickinson
CBA Analysis software
Hood for cell culture
Incubator for cell culture
Centrifuge
Pipettes

3 Materials and Reagents
Buffy coat
DMEM GIBCO
Human serum type AB SIGMA
L-Glutamine GIBCO
Penicillin-Streptomycin GIBCO
Ficoll PHARMACIA
96 well microtiter plate for cell culture COSTAR
Concanavalin A SIGMA
Human Th1/Th2 Cytokine CBA Kit Becton-Dickinson
PBS GIBCO
FALCON 50 ml sterile Becton-Dickinson
BSA SIGMA
Glycerol MERCK
DMSO SIGMA
96 well microtiter plate conical bottom NUNC

4 Method

4.1 Purification of Human PBMC from a Buffy Coat

The buffy coat 1 to 2 is diluted with DMEM. 25 ml of diluted blood is thereafter slowly added onto a 15 ml layer of Ficoll in a 50 ml Falcon tube, and tubes are centrifuged (2000 rpm, 20 min, at RT without brake). The interphase (ring) is then collected and the cells are washed with 25 ml of DMEM followed by a centrifuge step (1200 rpm, 5 min). This procedure is repeated three times. A buffy coat gives approximately 600×10⁶ total cells.

4.2 Screening

80 μl of 1.25×10⁶ cells/ml are diluted in DMEM+2.5% Human Serum+1% L-Glutamine+1% Penicilliin-Streptomycin and thereafter added to a 96 well microtiter plate.

10 μl are added per well (one condition per well): Proteins are diluted in PBS+20% Glycerol (the final dilution of the proteins is 1/10).

10 μl of the ConA Stimuli are then added per well (one condition per well):
ConA 50 μg/ml (the final concentration of ConA is 5 μg/ml)

After 48 h, cell supernatants are collected and human cytokines are measured by Human Th1/Th2 Cytokine CBA Kit Becton-Dickinson.

4.3 CBA Analysis

(for more details, refer to the booklet in the CBA kit)

i) Preparation of Mixed Human Th1/Th2 Capture Beads

The number of assay tubes that are required for the experiment is determined.

Each capture bead suspension is vigorously vortexed for a few seconds before mixing. For each assay to be analysed, 10 μl aliquot of each capture bead are added into a single tube labelled “mixed capture beads”. The Bead mixture is thoroughly vortexed.

ii) Preparation of Test Samples

Supernatants were diluted (1:4) using the Assay Diluent (20 μl of supernatants+60 μl of Assay Diluent). The sample dilution is then mixed before transferring samples into a 96 wells microtiter plate conical bottom (Nunc).

iii) Human Th1/Th2 Cytokine CBA Assay Procedure

50 μl of the diluted supernatants are added into a 96 wells microtiter plate conical bottom (Nunc). 50 μl of the mixed capture beads are added followed by 50 μl addition of the Human Th1/Th2 PE Detection Reagent. The plate is then incubated for 3 hours at RT and protected from direct exposure to light followed by centrifugation at 1500 rpm for 5 minutes. The supernatant is then carefully discarded. In a subsequent step, 200 μl of wash buffer are twice added to each well, centrifuged at 1500 rpm for 5 minutes and supernatant carefully discarded. 130 μl of wash buffer are thereafter added to each well to resuspend the bead pellet. The samples are finally analysed on a flow cytometer. The data are analysed using the CBA Application Software, Activity Base and Microsoft Excel software.

From the read-out of the assay it can be evaluated whether in-vitro, the protein of the invention has a consistent inhibitory effect on all cytokines tested (IFN-γ, TNF-α, IL-2, IL-4, IL-5, IL-10).

Moreover, based on the EC50 value, it can be easily evaluated which is the best inhibited cytokine and then arrive at the specific auto-immune/inflammatory disease, which is known to be linked to such cytokine particularly.

### Example 7

### Autoimmunity/Inflammatory Assays

Assays targeting T lymphocyte responses

Fast-Ligand-induced T cell death. This assay will reveal new modulators of receptor mediated cell death.

In this assay, T cell apoptosis is induced by stimulating Jurkat cells (a human T cell line) with recombinant 6 Histidine-tagged Fas Ligand combined with a monoclonal anti 6-his antibody. Death is quantified by release of LDH, a cytoplasmic enzyme released in the culture medium when cells are dying. The read out is a calorimetric assay read at 490 nm. T cells have been shown to be pathogenic in many autoimmune diseases, being able to control antigen-specific T cell death is a therapeutic strategy (e.g. anti-TNFα treatment in patient with Crohn's disease).

Human-MLR: proliferation and cytokine secretion. This cell-based assay measures the effects of novel proteins on lymphocyte proliferation and cytokine secretion or inhibition upon stimulation by PBMC from another donor (alloreactivity). These assay address antigen-specific T cell and antigen presenting cell functions, which are crucial cellular responses in any autoimmune diseases. Secreted cytokine (IL-2, 4, 5, 10, TNF-α and IFN-γ) are quantified by CBA.

Note: proliferation and cytokine secretion are independent responses.

Mouse-MLR: proliferation. This cell-based assay measures the effects of novel proteins on lymphocyte proliferation or inhibition of mouse spleen cells following stimulation by spleen cells from another donor (mouse strain). This cell-based assay measures the effect of novel proteins on T lymphocyte and antigen presenting cell responses and will be used to confirm activity of positives and hits identify in the h-MLR assays. This assay will be use to select proteins that will be tested in murine model of human diseases.

Human PBMC stimulated with the superantigen, TSST. Superantigens are strong modulators of the immune system affecting T cells. Superantigens influence immunologically mediated disorders such as IBD, inflammatory skin diseases like atopic dermatitis and psoriasis. In this cellular assay, we are specifically targeting T lymphocyte activation via the TCR but with different requirements than the T cell response to classical antigens, in particular in respect to co-stimulatory molecules.

Human PBMC stimulated with either ConA or PHA. These cell-based assays measure the effects of novel proteins on cytokine secretion induced by two different stimuli acting on different cells as measured by a cytokine bead array (CBA) assay (IL-2, IFN-γ, TNF-α, IL-5, IL-4 and IL-10).

Most of cytokines can have dual actions, pro or anti-inflammatory, depending of the injury, milieu and cellular target. Any protein with the capability to modulate cytokine secretion may have a therapeutic potential (e.g. decreasing IFN-γ and TNF-α would be beneficial in Th1-mediated autoimmune disease in contrast decreasing IL-4, IL-5 may be beneficial in Th2-mediated-diseases, inducing IL-10 would interesting in MS and SLE).

Assays Targeting Monocyte/Macrophages and Granulocyte Responses

Human PBMC stimulated with LPS. This cell-based assay measures the effects of novel proteins on cytokine secretion (IFN-γ, TNF-α) induced by LPS acting on monocytes/macrophages and granulocytes.

Any protein with the capability to modulate IFN-γ and TNF-α secretion would be beneficial in Th1-mediated autoimmune diseases.

Assays Targeting Neutrophil Responses

Neutrophils are important in inflammation and autoimmune diseases such as Rheumatoid Arthritis. Leukocyte chemo-attractants such as IL-8 initiate a sequence of adhesive interactions between cells and the micro-vascular endothelium, resulting in activation, adhesion and finally migration of neutrophils. The tissue infiltration of neutrophils depends on a reorganisation of cytoskeleton elements associated with specific changes in cell morphology of these cells.

This cell-based assay measures the effect of novel proteins on cytoskeleton reorganization of human neutrophils.

Assays Targeting B Lymphocyte Responses

Autoantibodies as well as infiltrating B cells are thought to be important in the pathogenesis of various autoimmune diseases, such as systemic lupus erithematosus (SLE), rheumatoid arthritis (RA), Sjogren's syndrome and myasthenia gravis. Compelling evidence indicates that a disregulation in B cell homeostasis could affect immune tolerance leading to the inappropriate survival of autoreactive B cells producing pathogenic antibodies and sustained inflammation. The identification of new factors that play critical roles in the regulation of B cell proliferation, survival and differentiation following B cell receptor triggering are of high relevance in the development of novel therapies.

B cell proliferation. This cell-based assay measures the effect of novel proteins on B cell survival.

B cell co-stimulation. This cell-based assay measures the effect of novel proteins on B cell co-stimulation.

Assays Targeting Monocvtes and Microglial Responses

THP-1 calcium flux. The Ca⁺-flux in THP1-cell assay measures the effects of novel proteins on their ability to trigger an intracellular calcium release (a generic second messenger events) from the endoplasmic reticulum.

Microglia cell proliferation (will be presented to the next IAC).

During proliferation of microglial progenitors, a number of colony-stimulating factors, including some cytokines, are known to play key roles. Among them, M-CSF is crucial for the final step of maturation of macrophages/microglia and is not replaceable by any other factor. The evaluation of this biological response may represent a way to influence the microglial activity and therefore an opportunity to identify molecules with therapeutic potential fro MS.

A cell-based assay was developed to measure the proliferative response of a microglia cell line to M-CSF. The feasibility and the robustness phases showed optimal results. This assay is in 96 well plates; non-radioactive substrate is required, easily automated.

### Example 8

### Neurological Assays Suitable for Exploration of the Biological Relevance of proteins Function

A number of neurological assays have been developed by the Applicant and are of use in the investigation of the biological relevance of protein function. Examples of neurological assays that have been developed by the Applicant include four types of assays. These are discussed below.

i. Oligodendrocytes-Based Assays

Oligodendrocytes are responsible for myelin formation in the CNS. In multiple sclerosis they are the first cells attacked and their loss leads to major behavioral impairment. In addition to curbing inflammation, enhancing the incomplete remyelination of lesions that occurs in MS has been proposed as a therapeutic strategy for MS. Like neurons, mature oligodendrocytes do not divide but the new oligodendrocytes can arise from progenitors. There are very few of these progenitor cells in adult brain and even in embryos the number of progenitor cells is inadequate for HTS.

Oli-neu is a murine cell line obtained by an immortalization of an oligodendrocyte precursor by the t-neu oncogene. They are well studied and accepted as a representative cell line to study young oligodendrocyte biology.

These cells can be used in two types of assays.

One, to identify factors stimulating oligodendrocytes proliferation, and the other to find factors promoting their differentiation. Both events are key in the perspective of helping renewal and repairing demyelinating diseases.

Another possible cell line is the human cell line, M03-13. M03-13 results from the fusion of rabdo-myosarcoma cells with adult human oligodendrocytes. However these cells have a reduced ability to differentiate into oligodendrocytes and their proliferating rate is not sufficient to allow a proliferation assay. Nevertheless, they express certain features of oligodendrocytes and their morphology is well adapted to nuclear translocation studies. Therefore this cell line can be used in assays based on nuclear translocation of three transcription factors, respectively NF-kB, Stat-1 and Stat-2. The Jak/Stats transcription pathway is a complex pathway activated by many factors such as IFN α, β, γ, cytokines (e.g. IL-2, IL6; IL-5) or hormones (e.g. GH, TPO, EPO). The specificity of the response depends on the combination of activated Stats. For example, it is noticeable that IFN-β activates Stat1, 2 and 3 nuclear translocations meanwhile IFN-γ only activates Stat1. In the same way, many cytokines and growth factors induced NF-kB translocation. In these assays the goal is to get a picture of activated pathways for a given protein.

ii. Astrocvtes-Based Assays

The biology of astrocytes is very complex, but two general states are recognized. In one state called quiescent, astrocytes regulate the metabolic and excitatory level of neurons by pumping glutamate and providing energetic substratum to neurons and oligodendrocytes. In the activated state, astrocytes produce chemokines and cytokines as well as nitric oxide. The first state could be considered as normal healthy while the second state occurs during inflammation, stroke or neurodegenerative diseases. When this activated state persists it should be regarded as a pathological state.

Many factors and many pathways are known to modulate astrocyte activation. In order to identify new factors modulating astrocyte activation U373 cells, a human cell line of astroglioma origin, can be used. NF-kB, c-Jun as well as Stats are signaling molecules known to play pivotal roles in astrocyte activation.

A series of screens based on the nuclear translocation of NF-κB, c-Jun and Stat1, 2 and 3 can be carried out. Prototypical activators of these pathways are IL-1b, IFN-beta or IFN-gamma. The goal is to identify proteins that could be used as therapeutics in the treatment of CNS diseases.

C. Neurons-Based Assays

Neurons are very complex and diverse cells but they have all in common two things. First they are post-mitotic cells, secondly they are innervating other cells. Their survival is linked to the presence of trophic factors often produced by the innervated target cells. In many neurodegenerative diseases the lost of target innervation leads to cell body atrophy and apoptotic cell death. Therefore identification of trophic factors supplementing target deficiency is very important in treatment of neurodegenerative diseases.

In this perspective a survival assay using NS1 cells, a sub-clone of rat PC12 cells, can be performed. These cells have been used for years and a lot of neurobiology knowledge has been first acquired on these cells before being confirmed on primary neurons including the pathways involved in neuron survival and differentiation (MEK, P13K, CREB). In contrast the N2A cells, a mouse neuroblastoma, are not responding to classical neurotrophic factors but Jun-kinase inhibitors prevent apoptosis induced by serum deprivation. Therefore assays on these two cell lines will help to find different types of “surviving promoting” proteins.

It is important to note that in the previous assays we will identify factors that promote both proliferation and differentiation. In order to identify factors specifically promoting neuronal differentiation, a NS 1 differentiation assay based on neurite outgrowth can be used. Promoting axonal or dendritic sprouting in neurodegenerative diseases could be advantageous for two reasons. It will first help the degenerating neurons to re-grow and re-establish a contact with the target cells. Secondly, it will potentiate the so-called collateral sprouting from healthy fibers, a compensatory phenomenon that delays terminal phases of neurodegenerative such as Parkinson or AD.

1. Endothelial Cells-Based Assays

The blood brain barrier (BBB) between brain and vessels is responsible of differences between cortical spinal fluid and serum compositions. The BBB results from a tight contact between endothelial cells and astrocytes. It maintains an immunotolerant status by preventing leukocytes penetration in brain, and allows the development of two parallels endocrine systems using the same intracellular signaling pathways. However, in many diseases or traumas, the BBB integrity is altered and leukocytes as well as serum proteins enter the brain inducing neuroinflammation. There is no easy in vitro model of BBB, but cultures of primary endothelial cells such as human embryonic umbilical endothelial cells (HUVEC) could mimic some aspect of BBB biology. For example, BBB leakiness could be induced by proteins stimulating intracellular calcium release. In the perspective of identifying proteins that modulate BBB integrity, a calcium mobilization assay with or without thrombin can be performed on HUVEC.

### Example 9

### Fibroblast Assays Suitable for Exploration of the Biological Relevance of the Protein Function

A number of fibroblasts assays have been developed by the Applicant and are of use in the investigation of the biological relevance of protein function. Examples of fibroblasts assays that have been developed by the Applicant include eight types of assays. These are discussed below.

Activation and pathological proliferation of fibroblasts are the key steps leading to a phenotype known as fibrosis. Fibrosis is characterized by the excessive deposition of extracellular matrix, especially collagen. Stromal cells, including fibroblasts, express specific pro- and anti-fibrotic proteins. Keratinocyte growth factor (KGF) is a well-characterized anti-fibrotic molecule. Additionally, oxidative damage and pro-inflammatory stimuli have been proposed to be among major events leading to myofibroblast phenotype and eventually to fibrosis. NF-kB is a mediator of oxidative stress and inflammatory reactions. Based on fibroblast biology, we have developed four cell-based assays, namely fibroblast proliferation, collagen production, NF-kB activation and KGF production assays.

A. Human Fibroblast Proliferation Assay

An activation and pathological proliferation of fibroblasts are the key steps leading to a phenotype known as fibrosis. The assay is based on fluorescence enhancement mediated by CyQUANT GR dye bound to cellular nucleic acids and measures the proliferative responses of human skin-derived fibroblasts to novel proteins and small molecules.

B. Type I Collagen Production by Human Fibroblasts

Fibrosis is characterized by the excessive deposition of extracellular matrix, especially collagen. Over production of type I collagen is the main manifestation of systemic sclerosis. TGFβ is known to up-regulate production of collagen in vitro and in vivo. We developed cell-based assay in order to test the ability of novel pro-or anti-fibrotic molecules to modulate basal or TGFβ1-stimulated levels of type I collagen production by human skin-derived fibroblasts.

C. Keratinocvte Growth Factor KGF) Production by Human Fibroblasts

KGF is an important mediator of stroma-to epithelium interactions in many organs (lung, pancreas, kidney, prostate, mammary, gland, uterus) during normal and pathological growth and development. KGF is specifically produced by stromal cells and its receptor is specifically expressed by epithelial cells. It is proposed that KGF might be an important player during pathophysiological reactions in fibrosis and thus can be used as a marker of these reactions. A KGF ELISA assay has been developed and using human lung-derived fibroblasts it has been shown that the KGF production can be significantly up-regulated by IL-1β and TNFα and down-regulated by TGFβ. These cytokines will be used as reference molecules in screening for novel proteins capable to induce KGF production.

D. NF-κB Transcription Activation in Fibroblasts

Oxidative damage and pro-inflammatory stimuli have been proposed to be among major events leading to myofibroblast phenotype and eventually to fibrosis. NF-κB is a mediator of oxidative stress and inflammatory reactions. Swiss 3T3 fibroblasts were generated with a stably integrated NF-κB-SEAP (secreted alkaline phosphatase) construct. NF-κB-SEAP is designed to measure the binding of transcription factors to the κ enhancer allowing a direct measurement of activation of the NF-κB pathway. The SEAP enzyme is secreted into the culture medium, so samples can be collected at various time points to assay for transcription activity without harvesting cells. The Swiss 3T3-NF-κB-SEAP cell line can be used as a cell-based assay to test novel Functional Genomics proteins and is very promising for testing small molecules, especially those with predicted pro-/anti-inflammatory activity.

E. Connective Tissue Growth Factor (CTGF) Promoter Activation/Repression in Fibroblasts

CTGF, a 38-kD cysteine-rich protein, stimulates the production of extracellular matrix elements by fibroblasts. CTGF overexpression has reportedly been found in many fibrotic human tissues, including lung, skin, liver, kidney and blood vessels. In vitro, TGFβ activates CTGF gene transcription in human lung fibroblasts. A CTGF promoter-reporter was constructed with secreted alkaline phosphatase (SEAP) as a reporter and Swiss 3T3 fibroblasts were generated with a stably integrated CTGF-SEAP construct. Using these fibroblasts it was shown that CTGF promoter is down-regulated by SARP-1, OPG and FSH and up-regulated by TGFβ.

F. KL-6 Production

KL-6, originally discovered as a pulmonary adenocarcinoma-related protein and later referred to as MUC-1, is a high-molecular-weight glycoprotein, now classified as Cluster 9 antigen. KL-6 is elevated in both sera and BALF of patients with idiopathic pulmonary fibrosis (IPF) and other lung interstitial diseases. In lung tissue from patients suffering from IPF, the majority of cells labelled with KL-6 antibodies are regenerating type II pneumocytes. Two peptides were designed to produce polyclonal antibodies against KL-6. KL-6 ELISA can be used to measure KL-6 production by human lung-derived type II pneumocytes.

G. Neutralization of Apoptosis of L-929 Fibroblasts Treated with Soluble Recombinant TRAIL (TNF-Related Anoptosis-Inducing Ligand)

TRAIL has been shown to be one of the cellular ligands for osteoprotegerin (OPG). This assay can be used to measure the biological activity of OPG.

H. RANKL (Receptor Activator of NF-IcB Ligand) Production by Human Fibroblasts

RANKL is another ligand for OPG. This assay can also be used to measure the biological activity of OPG.

### Example 10

### Reproductive Health Assays Suitable for Exploration of the Biological Relevance of Proteins Function

A number of reproductive health-related assays have been developed by the Applicant and are of use in the investigation of the biological relevance of SCS0010 protein function. In view of the probable implication of SCS0010 in male infertility (see therapeutic uses), such assays seem of particular relevance. Examples of reproductive health-related assays that have been developed by the Applicant include 18 cell-based assays for reproductive health. These are discussed below.

1. Primary Human Uterine Smooth Muscle Proliferation Assay:

The proliferation of uterine smooth muscle cells is a precursor for development of tumors in uterine fibroid disease in women. In this assay, the goal is to identify proteins that inhibit proliferation of primary human uterine smooth muscle cells.

2. JEG-3 Implantation Assay:

JEG-3 cells are a choriotrophoblastic human cancer cell line used as a model for the blastocyst during implantation. Ishikawa cells are a relatively non-differentiated endometrial human cancer cell line that is used as a model for the decidua. JEG-3 cells will “implant” into human decidual tissue. In this assay, a 2-chamber system is used where fluorescently labeled JEG-3 cells invade through a Matrigel-coated porous membrane from an upper chamber into a lower chamber when Ishikawa cells or Ishikawa-conditioned medium are placed into the lower chamber. The cells that migrate are quantified in a plate reader. The goal is to identify proteins that increase invasion of JEG-3 cells for use in aiding implantation in vivo.

C. Osteopontin Bead Assa (Ishikawa cells):

Ishikawa human endometrial cancer cells are used as a model for implantation. At the time of implantation in the human, various integrins are expressed by the uterine endometrium that is thought to bind to proteins expressed by the blastocyst. Ishikawa cells have been shown in the literature to express avb3, which is the integrin expressed by the uterine endometrium during the “window of implantation”. This integrin is believed to bind the osteopontin expressed by the trophoblast. In this assay, osteopontin-coated fluorescent beads represent the blastocyst, and the Ishikawa cells are primed to accept them for binding by treating them with estradiol. The goal is to identify proteins that increase the ability of the Ishikawa cells to bind the osteopontin-beads as an aid to increase receptivity of the uterine endometrium at the time of implantation.

D. HuF6 Assay:

HuF6 cells are primary human uterine fibroblast cells. These cells can be induced to decidualize by treating them with IL-1β. A marker for decidualization is production of PGE2, which is measured by ELISA. The goal is to identify proteins that increase production of PGE2 by the HuF6 cells as a way of enhancing decidualization during early pregnancy.

2. Endometriosis Assay:

Peritoneal TNFα plays a role in endometriosis by inducing the sloughed endometrial cells from the uterus to adhere to and proliferate on peritoneal mesothelial cells. In this assay, BEND cells are treated with TNFα, which increases their ability to bind fibronectin-coated fluorescent beads as an assay for adherence during endometriosis. The goal is to identify proteins that decrease or inhibit the ability of TNFα to stimulate bead-binding capacity of the cells.

F. Cyclic AMP Assay Using JC-410 Porcine Granulose Cells Stably Transfected with hLHR:

In Polycystic Ovary Syndrome, LH from the pituitary is relatively high, and induces androgen output from the ovarian thecal cells. This assay is used to look for an inhibitor of LH signaling which could be used to decrease the action of LH at the ovary during PCOS. The JC-410 porcine granulosa cell line is stably transfected with the human LH receptor. Treatment with LH results in cAMP production.

G. Cyclic AMP Assay using JC-410 Porcine Granulose Cells Stably Transfected with HFSHR:

The JC-410 porcine granulosa cell line was stably transfected with the human FSHR. Treatment with FSH stimulates cAMP production, which is measured in this assay. The goal is to identify proteins that enhance FSH action in the granulosa cells.

H. LbetaT2 (Mouse) Pituitary Cells Assay:

The LbetaT2 is an immortalized murine pituitary gonadotroph cell line. Stimulation with Activin alone or with GnRH+Activin results in secretion of FSH (stimulation with GnRH alone results in secretion of LH.) The cells can either be treated with GnRH+Bioscreen proteins to find proteins that act in concert with GnRH to stimulate FSH production, or they can be treated with Bioscreen proteins alone to find a protein that can stimulate FSH secretion like activin alone.

I. Cumulus Expansion Assay:

The cumulus-expansion assay using murine cumulus-oocyte complexes (2/well) has been validated in a 96-well format to assay for proteins that affect oocyte maturation (measured by cumulus expansion). Two 96-well plates can be processed per assay, and 2 assays per week can be performed. If Bioscreen proteins are assayed at only one concentration, all Bioscreen I proteins can be assayed in a month. The read-out may be a yes/no answer for expansion, or image analysis programs may be used to measure expansion in a quantitative manner.

J. RWPE Proliferation Assay:

Benign prostatic hyperplasia is characterized by growth of prostatic epithelium and stroma that is not balanced by apoptosis, resulting in enlargement of the organ. RWPE is a regular human prostatic epithelial cell line that was immortalized with the HPV-18, and may be used in place of primary human prostatic epithelial cells.

K. HT-1080 Fibrosarcoma Invasion Assay:

This assay was developed as a positive cell control for the JEG-3 implantation assay (above). This is a well-established assay as a model for cancer metastasis. Fluorescently-labeled HT-1080 human fibrosarcoma cells are cultured in the upper chamber of a 2-chamber system, and can be stimulated to invade through the porous Matrigel-coated membrane into the bottom chamber where they are quantified. The goal is to identify a protein that inhibits the invasion. The cells are stimulated to invade by adding serum to the bottom chamber and are inhibited with doxycycline.

L. Primary Human Uterine Smooth Muscle Assay:

One of the hallmarks of uterine fibroid disease is collagen deposition by the uterine smooth muscle cells that have become leioymyomas. Primary human uterine smooth muscle cells are stimulated to produce collagen by treatment with TGFβ, which is blocked with Rebif. The goal is to discover proteins that inhibit this fibrotic phenotype.

M. Human Leiomyoma Cells Proliferation Assay:

A human leiomyoma cell line may be used as a model for uterine fibroid disease in a proliferation assay. The cells grow very slowly and we are stimulating them to grow at a faster rate by treating them with estradiol and growth factors. The goal is to identify proteins that inhibit estradiol-dependent growth of leiomyoma cells.

N. 937 Migration Assay:

Endometriotic lesions secrete cytokines that recruit immune cells to the peritoneal cavity. These immune cells (especially activated macrophages and T lymphocytes) mediate inflammatory symptoms that are common to endometriosis. RANTES has been shown to be produced by endometriotic stromal cells and is present in the peritoneal fluid. In this assay, U937, a monocytic cell line used as a model for activated macrophages, can be induced by treating the lower level of a 2-chamber culture system to migrate from the upper chamber. If the cells are pre-loaded with fluorescent dye, they can be quantified in the lower chamber. The goal is to identify proteins that inhibit the migration of the U937 cells.

O. JEG3 Human Trophoblast Assay:

The trophoblast of the blastocyst produces HLA-G, a class I HLA molecule that is believed to be important in preventing immunological rejection of the embryo by the mother. During pre-eclampsia, HLA-G levels are low or non-existent, presumably resulting in hallmark symptoms such as poor invasion of the trophoblast into the endometrium and spiral arteries because of maternal immunological interference. The JEG-3 human trophoblast cell line produces HLA-G, which can be increased by treatment with IL-10 or LIF. An ELISA can be used to measure HLA-G production by JEG-3 cells, with the goal being the discovery of other proteins that can increase HLA-G production.

P. Primary Rat Ovarian Dispersate Assay:

Due to the difficulties in measuring appreciable amounts of steroids from the JC-410-FSHR/LHR cell lines, an assay using primary cells from whole ovaries taken from immature rats has been developed. Initially, estradiol production from these cultures is measured after treatment with FSH and/or LH. The goal is then to identify proteins that enhance gonadotropin-stimulated steroidogenesis, or proteins that work alone to increase steroidogenesis by these cultures.

Q. Mouse IVF Assay:

In this assay, sperm function, measured by ability to fertilize oocytes, is assayed with the goal of finding proteins that stimulate fertilizing potential of sperm.

R. Primary Human Prostate Stromal Cells Proliferation Assay:

An assay for the epithelial component of BPH has already been described above (see RWPE assay above). This assay uses primary human prostate stromal cells as a model for proliferation of these cells during BPH. The goal is to identify proteins that inhibit proliferation of these cells.

### REFERENCES

Andersen D C and Krummen L, Curr. Opin. Biotechnol., 13: 117-23, 2002.
Baker K N et al., Trends Biotechnol, 20: 149-56, 2002.
Blagoev B and Pandey A, Trends Biochem Sci., 26: 639-41, 2001.
Bock A, Science, 292: 453-4, 2001.
Bunz F, Curr. Opin. Oncol., 14: 73-8, 2002.
Burgess R R and Thompson N E, Curr. Opin. Biotechnol., 12: 450-4, 2001.
Chambers S P, Drug Disc. Today, 14: 759-765, 2002.
Chu L and Robinson D K, Curr. Opin. Biotechnol., 13: 304-8, 2001.
Cleland J L et al., Curr. Opin. Biotechnol., 12: 212-9, 2001.
Coleman R A et al., Drug Discov. Today, 6: 1116-1126, 2001.
Constans A, The Scientist, 16(4): 37, 2002.
Davis B G and Robinson M A, Curr. Opin. Drug Discov. Devel., 5: 279-88, 2002.
Dougherty D A, Curr. Opin. Chem. Biol., 4: 645-52, 2000.
Garnett M C, Adv. Drug. Deliv. Rev., 53: 171-216, 2001.
Gavilondo J V and Larrick J W, Biotechniques, 29: 128-136, 2000.
Gendel S M, Ann. NY Acad. Sci., 964: 87-98, 2002.
Giddings G, Curr. Opin. Biotechnol., 12: 450-4, 2001.
Golebiowski A et al., Curr. Opin. Drug Discov. Devel., 4: 428-34, 2001.
Gupta P et al., Drug Discov. Today, 7: 569-579, 2002.
Haupt K, Nat. Biotechnol., 20: 884-885, 2002.
Hruby V J and Balse P M, Curr. Med. Chem, 7: 945-70, 2000.
Johnson D E and Wolfgang G H, Drug Discov. Today, 5: 445-454, 2000.
Kane J F, Curr. Opin. Biotechnol, 6: 494-500, 1995.
Kolb A F, Cloning Stem Cells, 4: 65-80, 2002.
Kuroiwa Y et al., Nat. Biotechnol, 20: 889-94, 2002.
Lin Cereghino G P et al., Curr. Opin. Biotechnol, 13: 329-332, 2001.
Lowe C R et al., J. Biochem. Biophys. Methods, 49: 561-74, 2001.
Luo B and Prestwich G D, Exp. Opin. Ther. Patents, 11: 1395-1410, 2001.
Mulder N J and Apweiler R, Genome Biol., 3(1):REVIEWS2001, 2002
Nlsson J et al., Protein Expr. Purif., 11: 1-16, 1997.
Pearson W R and Miller W, Methods Enzymol., 210: 575-601, 1992.
Pellois J P et al., Nat. Biotechnol, 20: 922-6, 2002.
Pillai 0 and Panchagnula R, Curr. Opin. Chem. Biol., 5: 447-451, 2001
Rehm B H, Appl. Microbiol. Biotechnol., 57: 579-92, 2001.
Robinson C R, Nat. Biotechnol., 20: 879-880, 2002.
Rogov S I and Nekrasov A N, Protein Eng., 14: 459-463, 2001.
Schellekens H, Nat. Rev. Drug Discov., 1: 457-62, 2002
Sheibani N, Prep. Biochem. Biotechnol., 29: 77-90, 1999.
Stevanovic S, Nat. Rev. Cancer, 2: 514-20, 2002.
Templin M F et al., Trends Biotechnol., 20: 160-6, 2002.
Tribbick G, J. Immunol. Methods, 267: 27-35, 2002.
van den Burg B and Eij sink V, Curr. Opin. Biotechnol., 13: 333-337, 2002.
van Dijk M A and van de Winkel J G, Curr. Opin. Chem. Biol., 5: 368-74, 2001.
Villain M et al., Chem. Biol., 8: 673-9, 2001.

1-41. (canceled)

42. A composition of matter comprising:
(a) an isolated polypeptide having IL-8-like/TMEM9 activity selected from the group consisting of:
(1) the amino acid sequence of SEQ ID NO: 2;
(2) the mature form of SEQ ID NO: 2 (SEQ ID NO:4 or SEQ ID NO: 7);
(3) the histidine tag form of SEQ ID NO: 4 (SEQ ID NO:5);
(4) active variants of SEQ ID NO: 2, wherein any amino acid specified in the chosen sequence is non-conservatively substituted, provided that no more than 15% of the amino acid residues in the sequence are so changed; or
(5) the active fragment, precursor, salt, or derivative of the amino acid sequences given in (1) to (3);
(b) an isolated polypeptide having IL-8-like/TMEM9 activity that is a naturally occurring allelic variant of SEQ ID NO: 2 or SEQ ID NO:4 or SEQ ID NO: 5 or SEQ ID NO: 7;
(c) an isolated polypeptide having IL-8-like/TMEM9 activity that is a naturally occurring allelic variant of SEQ ID NO: 2 or SEQ ID NO:4 or SEQ ID NO: 5 or SEQ ID NO: 7, wherein the variant is the translation of a single nucleotide polymorphism;
(d) a fusion protein comprising a polypeptide according to (a) or (b) or (c);
(e) a fusion protein comprising a polypeptide according to (a) or (b) or (c), wherein said fusion protein comprises one or more amino acid sequence selected from: membrane-bound protein, immunoglobulin constant region, multimerization domains, extracellular proteins, signal peptide-containing proteins, export signal-containing proteins;
(f) an antagonist of a polypeptide according to (a) or (b) or (c), wherein said antagonist comprises an amino acid sequence containing the non-conservative substitution and/or the deletion of one or more residues in the corresponding polypeptide;
(g) an isolated ligand which binds specifically to a polypeptide according (a) or (b) or (c);
(h) an isolated ligand which binds specifically to a polypeptide according (a) or (b) or (c) and that antagonizes or inhibits the IL-8-like/TMEM9 activity of said polypeptide;
(i) an isolated ligand which binds specifically to a polypeptide according (a) or (b) or (c), antagonizes or inhibits the IL-8-like/TMEM9 activity of said polypeptide, and is selected from a monoclonal antibody, a polyclonal antibody, a humanized antibody, an antigen binding fragment, or the extracellular domain of a membrane-bound protein;
(j) an isolated polypeptide as set forth in (a) or (b) or (c) or (d) or (e) conjugated or complexed with a molecule selected from radioactive labels, fluorescent labels, biotin, or cytotoxic agents;
(k) a peptide mimetic designed on the sequence and/or the structure of a polypeptide according (a) or (b) or (c) or (d) or (e);
(l) an isolated nucleic acid encoding for an isolated polypeptide as set forth in (a) or (b) or (c) or (d) or (e) or (f);
(m) an isolated nucleic acid comprising SEQ ID NO: 1 or SEQ ID NO: 3 or SEQ ID NO: 6, or the complement of said sequence;
(n) a purified nucleic acid which hybridizes under high stringency conditions or exhibits at least about 85% identity over a stretch of at least about 30 nucleotides with a nucleic acid comprising SEQ ID NO: 1 or SEQ ID NO: 3 or SEQ ID NO: 6, or a complement of said sequence;
(O) a vector comprising a nucleic acid as set forth in (l) or (m) or (n);
(p) a vector comprising a nucleic acid as set forth in (1) or (m) or (n) operatively linked to expression control sequences allowing expression in prokaryotic or eukaryotic host cells of the encoded polypeptide;
(q) an isolated polypeptide encoded by a nucleic acid as set forth in (l) or (m) or (n);
(r) a host cell transformed with a vector or a nucleic acid as set forth in (l) or (m) or (n) or (O) or (p);
(s) a transgenic animal cell that has been transformed with a vector or a nucleic acid as set forth in (l) or (m) or (n) or (O) or (p);
(t) a transgenic non-human animal that has been transformed with a vector or a nucleic acid as set forth in (l) or (m) or (n) or (O) or (p);
(u) a compound that enhances the expression level of a polypeptide as set forth in (a) or (b) or (c) or (d) in a cell or in an animal;
(v) a compound that reduces the expression level of a polypeptide as set forth in (a) or (b) or (c) or (d) in a cell or in an animal;
(w) an antisense oligonucleotide or a small interfering RNA that reduces the expression level of a polypeptide as set forth in (a) or (b) or (c) or (d) in a cell or in an animal; or
(x) a composition comprising a carrier and:
(1) a polypeptide as set forth in (a) or (b) or (c) or (d) or (e) or (j) or (q);
(2) an antagonist or ligand as set forth in (f) or (g) or (h) or (i);
(3) a peptide mimetic as set forth in (k);
(4) a vector or isolated nucleic acid as set forth in (l) or (m) or (n) or (O) or (p);
(5) a cell as set forth in (r) or (s); or
(6) a compound as set forth in (u) or (v) or (w).

43. A method of using a composition of matter according to claim 42 for producing cells capable of expressing a polypeptide; making a polypeptide; the preparation of a pharmaceutical composition; the treatment or prevention of a disease needing an increase in the IL-8-like/TMEM9 activity; the therapy or in the prevention of a disease associated to the excessive IL-8-like/TMEM9 activity; screening candidate compounds effective to treat a disease related to the IL-8-like/TMEM9 polypeptides; identifying a candidate compound as an antagonist/inhibitor or agonist/activator of a polypeptide; determining the activity and/or the presence of the polypeptide; or determining the presence or the amount of a transcript or of a nucleic acid.

44. The method according to claim 43, wherein said method comprises a process for producing cells capable of expressing a polypeptide comprising genetically engineering cells with a vector or a nucleic acid comprising:
(a) an isolated nucleic acid encoding for an isolated polypeptide comprising an isolated polypeptide having IL-8-like/TMEM9 activity selected from the group consisting of:
(1) the amino acid sequence of SEQ ID NO: 2;
(2) the mature form of SEQ ID NO: 2 (SEQ ID NO:4 or SEQ ID NO: 7);
(3) the histidine tag form of SEQ ID NO: 4 (SEQ ID NO:5);
(4) active variants of SEQ ID NO: 2, wherein any amino acid specified in the chosen sequence is non-conservatively substituted, provided that no more than 15% of the amino acid residues in the sequence are so changed;
(5) the active fragment, precursor, salt, or derivative of the amino acid sequences given in (1) to (3);
(6) an isolated polypeptide having IL-8-like/TMEM9 activity that is a naturally occurring allelic variant of SEQ ID NO: 2 or SEQ ID NO:4 or SEQ ID NO: 5 or SEQ ID NO: 7;
(7) an isolated polypeptide having IL-8-like/TMEM9 activity that is a naturally occurring allelic variant of SEQ ID NO: 2 or SEQ ID NO:4 or SEQ ID NO: 5 or SEQ ID NO: 7, wherein the variant is the translation of a single nucleotide polymorphism;
(8) a fusion protein comprising a polypeptide according to (1) or (2) or (3);
(9) a fusion protein comprising a polypeptide according to (1) or (2) or (3), wherein said fusion protein comprises one or more amino acid sequence selected from: membrane-bound protein, immunoglobulin constant region, multimerization domains, extracellular proteins, signal peptide-containing proteins, export signal-containing proteins; or
(10) an antagonist of a polypeptide according to (1) or (2) or (3), wherein said antagonist comprises an amino acid sequence containing the non-conservative substitution and/or the deletion of one or more residues in the corresponding polypeptide;
(b) an isolated nucleic acid comprising SEQ ID NO: 1 or SEQ ID NO: 3 or SEQ ID NO: 6, or the complement of said sequence;
(c) a purified nucleic acid which hybridizes under high stringency conditions or exhibits at least about 85% identity over a stretch of at least about 30 nucleotides with a nucleic acid comprising SEQ ID NO: 1 or SEQ ID NO: 3 or SEQ ID NO: 6, or a complement of said sequence;
(d) a vector comprising a nucleic acid as set forth in (a) (or (b) or (c); or
(e) a vector comprising a nucleic acid as set forth in (a) or (b) or (c) operatively linked to expression control sequences.

45. The method according to claim 43, wherein said method comprises a method for making a polypeptide comprising culturing a cell comprising a vector or a nucleic acid comprising:
(a) an isolated nucleic acid encoding for an isolated polypeptide comprising an isolated polypeptide having IL-8-like/TMEM9 activity selected from the group consisting of:
(1) the amino acid sequence of SEQ ID NO: 2;
(2) the mature form of SEQ ID NO: 2 (SEQ ID NO:4 or SEQ ID NO: 7);
(3) the histidine tag form of SEQ ID NO: 4 (SEQ ID NO:5);
(4) active variants of SEQ ID NO: 2, wherein any amino acid specified in the chosen sequence is non-conservatively substituted, provided that no more than 15% of the amino acid residues in the sequence are so changed;
(5) the active fragment, precursor, salt, or derivative of the amino acid sequences given in (1) to (3);
(6) an isolated polypeptide having IL-8-like/TMEM9 activity that is a naturally occurring allelic variant of SEQ ID NO: 2 or SEQ ID NO:4 or SEQ ID NO: 5 or SEQ ID NO: 7;
(7) an isolated polypeptide having IL-8-like/TMEM9 activity that is a naturally occurring allelic variant of SEQ ID NO: 2 or SEQ ID NO:4 or SEQ ID NO: 5 or SEQ ID NO: 7, wherein the variant is the translation of a single nucleotide polymorphism;
(8) a fusion protein comprising a polypeptide according to (1) or (2) or (3);
(9) a fusion protein comprising a polypeptide according to (1) or (2) or (3), wherein said fusion protein comprises one or more amino acid sequence selected from: membrane-bound protein, immunoglobulin constant region, multimerization domains, extracellular proteins, signal peptide-containing proteins, export signal-containing proteins; or
(10) an antagonist of a polypeptide according to (1) or (2) or (3), wherein said antagonist comprises an amino acid sequence containing the non-conservative substitution and/or the deletion of one or more residues in the corresponding polypeptide;
(b) an isolated nucleic acid comprising SEQ ID NO: 1 or SEQ ID NO: 3 or SEQ ID NO: 6, or the complement of said sequence;
(c) a purified nucleic acid which hybridizes under high stringency conditions or exhibits at least about 85% identity over a stretch of at least about 30 nucleotides with a nucleic acid comprising SEQ ID NO: 1 or SEQ ID NO: 3 or SEQ ID NO: 6, or a complement of said sequence;
(d) a vector comprising a nucleic acid as set forth in (a) (or (b) or (c); or
(e) a vector comprising a nucleic acid as set forth in (a) or (b) or (c) operatively linked to expression control sequences;
under conditions in which the nucleic acid or vector is expressed, and recovering the polypeptide encoded by said nucleic acid or vector from the culture.

46. The method according to claim 43, wherein said method for the preparation of a pharmaceutical composition comprises combining a pharmaceutically acceptable carrier with:
(a) an isolated polypeptide having IL-8-like/TMEM9 activity selected from the group consisting of:
(1) the amino acid sequence of SEQ ID NO: 2;
(2) the mature form of SEQ ID NO: 2 (SEQ ID NO:4 or SEQ ID NO: 7);
(3) the histidine tag form of SEQ ID NO: 4 (SEQ ID NO:5);
(4) active variants of SEQ ID NO: 2, wherein any amino acid specified in the chosen sequence is non-conservatively substituted, provided that no more than 15% of the amino acid residues in the sequence are so changed;
(5) the active fragment, precursor, salt, or derivative of the amino acid sequences given in (1) to (3);
(b) an isolated polypeptide having IL-8-like/TMEM9 activity that is a naturally occurring allelic variant of SEQ ID NO: 2 or SEQ ID NO:4 or SEQ ID NO: 5 or SEQ ID NO: 7;
(c) an isolated polypeptide having IL-8-like/TMEM9 activity that is a naturally occurring allelic variant of SEQ ID NO: 2 or SEQ ID NO:4 or SEQ ID NO: 5 or SEQ ID NO: 7, wherein the variant is the translation of a single nucleotide polymorphism;
(d) a fusion protein comprising a polypeptide according to (a) or (b) or (c);
(e) a fusion protein comprising a polypeptide according to (a) or (b) or (c), wherein said fusion protein comprises one or more amino acid sequence selected from: membrane-bound protein, immunoglobulin constant region, multimerization domains, extracellular proteins, signal peptide-containing proteins, export signal-containing proteins;
(f) an antagonist of a polypeptide according to (a) or (b) or (c), wherein said antagonist comprises an amino acid sequence containing the non-conservative substitution and/or the deletion of one or more residues in the corresponding polypeptide;
(g) an isolated ligand which binds specifically to a polypeptide according (a) or (b) or (c);
(h) an isolated ligand which binds specifically to a polypeptide according (a) or (b) or (c) and that antagonizes or inhibits the IL-8-like/TMEM9 activity of said polypeptide;
(i) an isolated ligand which binds specifically to a polypeptide according (a) or (b) or (c), antagonizes or inhibits the IL-8-like/TMEM9 activity of said polypeptide, and is selected from a monoclonal antibody, a polyclonal antibody, a humanized antibody, an antigen binding fragment, or the extracellular domain of a membrane-bound protein;
(j) an isolated polypeptide as set forth in (a) or (b) or (c) or (d) or (e) conjugated or complexed with a molecule selected from radioactive labels, fluorescent labels, biotin, or cytotoxic agents;
(k) a peptide mimetic designed on the sequence and/or the structure of a polypeptide according (a) or (b) or (c) or (d) or (e);
(l) an isolated nucleic acid encoding for an isolated polypeptide as set forth in (a) or (b) or (c) or (d) or (e) or (f);
(m) an isolated nucleic acid comprising SEQ ID NO: 1 or SEQ ID NO: 3 or SEQ ID NO: 6, or the complement of said sequence;
(n) a purified nucleic acid which hybridizes under high stringency conditions or exhibits at least about 85% identity over a stretch of at least about 30 nucleotides with a nucleic acid comprising SEQ ID NO: 1 or SEQ ID NO: 3 or SEQ ID NO: 6, or a complement of said sequence;
(O) a vector comprising a nucleic acid as set forth in (l) or (m) or (n);
(p) a vector comprising a nucleic acid as set forth in (1) or (m) or (n) operatively linked to expression control sequences allowing expression in prokaryotic or eukaryotic host cells of the encoded polypeptide;
(q) an isolated polypeptide encoded by a nucleic acid as set forth in (l) or (m) or (n);
(r) a host cell transformed with a vector or a nucleic acid as set forth in (1) or (m) or (n) or (O) or (p);
(s) a transgenic animal cell that has been transformed with a vector or a nucleic acid as set forth in (l) or (m) or (n) or (O) or (p);
(t) a compound that enhances the expression level of a polypeptide as set forth in (a) or (b) or (c) or (d) in a cell or in an animal;
(u) a compound that reduces the expression level of a polypeptide as set forth in (a) or (b) or (c) or (d) in a cell or in an animal; or
(v) an antisense oligonucleotide or a small interfering RNA that reduces the expression level of a polypeptide as set forth in (a) or (b) or (c) or (d) in a cell or in an animal.

47. The method according to claim 43, wherein said method for the treatment or prevention of a disease needing an increase in the IL-8-like/TMEM9 activity comprises the administration of a therapeutically effective amount of:
(a) an isolated polypeptide having IL-8-like/TMEM9 activity selected from the group consisting of:
(1) the amino acid sequence of SEQ ID NO: 2;
(2) the mature form of SEQ ID NO: 2 (SEQ ID NO:4 or SEQ ID NO: 7);
(3) the histidine tag form of SEQ ID NO: 4 (SEQ ID NO:5);
(4) active variants of SEQ ID NO: 2, wherein any amino acid specified in the chosen sequence is non-conservatively substituted, provided that no more than 15% of the amino acid residues in the sequence are so changed;
(5) the active fragment, precursor, salt, or derivative of the amino acid sequences given in (1) to (3);
(b) an isolated polypeptide having IL-8-like/TMEM9 activity that is a naturally occurring allelic variant of SEQ ID NO: 2 or SEQ ID NO:4 or SEQ ID NO: 5 or SEQ ID NO: 7;
(c) an isolated polypeptide having IL-8-like/TMEM9 activity that is a naturally occurring allelic variant of SEQ ID NO: 2 or SEQ ID NO:4 or SEQ ID NO: 5 or SEQ ID NO: 7, wherein the variant is the translation of a single nucleotide polymorphism;
(d) a fusion protein comprising a polypeptide according to (a) or (b) or (c);
(e) a fusion protein comprising a polypeptide according to (a) or (b) or (c), wherein said fusion protein comprises one or more amino acid sequence selected from: membrane-bound protein, immunoglobulin constant region, multimerization domains, extracellular proteins, signal peptide-containing proteins, export signal-containing proteins;
(f) a peptide mimetic designed on the sequence and/or the structure of a polypeptide according (a) or (b) or (c) or (d) or (e);
(g) an isolated nucleic acid encoding for an isolated polypeptide as set forth in (a) or (b) or (c) or (d) or (e) or (f);
(h) an isolated nucleic acid comprising SEQ ID NO: 1 or SEQ ID NO: 3 or SEQ ID NO: 6, or the complement of said sequence;
(i) a purified nucleic acid which hybridizes under high stringency conditions or exhibits at least about 85% identity over a stretch of at least about 30 nucleotides with a nucleic acid comprising SEQ ID NO: 1 or SEQ ID NO: 3 or SEQ ID NO: 6, or a complement of said sequence;
(j) a vector comprising a nucleic acid as set forth in (l) or (m) or (n);
(k) a vector comprising a nucleic acid as set forth in (1) or (m) or (n) operatively linked to expression control sequences allowing expression in prokaryotic or eukaryotic host cells of the encoded polypeptide;
(l) an isolated polypeptide encoded by a nucleic acid as set forth in (1) or (m) or (n);
(m) a host cell transformed with a vector or a nucleic acid as set forth in (1) or (m) or (n) or (O) or (p);
(n) a transgenic animal cell that has been transformed with a vector or a nucleic acid as set forth in (l) or (m) or (n) or (O) or (p); or
(O) a compound that enhances the expression level of a polypeptide as set forth in (a) or (b) or (c) or (d) in a cell or in an animal.

48. The method according to claim 43, wherein said method for the treatment or prevention of diseases comprises the administration to an individual of a therapeutically effective amount of:
(a) an antagonist of a polypeptide an isolated polypeptide having IL-8-like/TMEM9 activity selected from the group consisting of:
(1) the amino acid sequence of SEQ ID NO: 2;
(2) the mature form of SEQ ID NO: 2 (SEQ ID NO:4 or SEQ ID NO: 7);
(3) the histidine tag form of SEQ ID NO: 4 (SEQ ID NO:5);
(4) active variants of SEQ ID NO: 2, wherein any amino acid specified in the chosen sequence is non-conservatively substituted, provided that no more than 15% of the amino acid residues in the sequence are so changed;
(5) the active fragment, precursor, salt, or derivative of the amino acid sequences given in (1) to (3);
(b) an isolated polypeptide having IL-8-like/TMEM9 activity that is a naturally occurring allelic variant of SEQ ID NO: 2 or SEQ ID NO:4 or SEQ ID NO: 5 or SEQ ID NO: 7;
(c) an isolated polypeptide having IL-8-like/TMEM9 activity that is a naturally occurring allelic variant of SEQ ID NO: 2 or SEQ ID NO:4 or SEQ ID NO: 5 or SEQ ID NO: 7, wherein the variant is the translation of a single nucleotide polymorphism;
wherein said antagonist of (a) or (b) or (c) comprises an amino acid sequence containing the non-conservative substitution and/or the deletion of one or more residues in the corresponding polypeptide;
(d) an isolated ligand which binds specifically to a polypeptide according (a) or (b) or (c);
(e) an isolated ligand which binds specifically to a polypeptide according (a) or (b) or (c) and that antagonizes or inhibits the IL-8-like/TMEM9 activity of said polypeptide;
(f) an isolated ligand which binds specifically to a polypeptide according (a) or (b) or (c), antagonizes or inhibits the IL-8-like/TMEM9 activity of said polypeptide, and is selected from a monoclonal antibody, a polyclonal antibody, a humanized antibody, an antigen binding fragment, or the extracellular domain of a membrane-bound protein;
(g) a compound that reduces the expression level of a polypeptide as set forth in (a) or (b) or (c) or (d) in a cell or in an animal; or
(h) an antisense oligonucleotide or a small interfering RNA that reduces the expression level of a polypeptide as set forth in (a) or (b) or (c) or (d) in a cell or in an animal.

49. The method according to claim 43, wherein said method for screening candidate compounds effective to treat a disease related to the IL-8-like/TMEM9 polypeptides comprises: contacting a cell, a transgenic animal cell, or a transgenic non-human animal comprising a vector or a nucleic acid comprising:
(a) an isolated nucleic acid encoding for an isolated polypeptide comprising an isolated polypeptide having IL-8-like/TMEM9 activity selected from the group consisting of:
(1) the amino acid sequence of SEQ ID NO: 2;
(2) the mature form of SEQ ID NO: 2 (SEQ ID NO:4 or SEQ ID NO: 7);
(3) the histidine tag form of SEQ ID NO: 4 (SEQ ID NO:5);
(4) active variants of SEQ ID NO: 2, wherein any amino acid specified in the chosen sequence is non-conservatively substituted, provided that no more than 15% of the amino acid residues in the sequence are so changed;
(5) the active fragment, precursor, salt, or derivative of the amino acid sequences given in (1) to (3);
(6) an isolated polypeptide having IL-8-like/TMEM9 activity that is a naturally occurring allelic variant of SEQ ID NO: 2 or SEQ ID NO:4 or SEQ ID NO: 5 or SEQ ID NO: 7;
(7) an isolated polypeptide having IL-8-like/TMEM9 activity that is a naturally occurring allelic variant of SEQ ID NO: 2 or SEQ ID NO:4 or SEQ ID NO: 5 or SEQ ID NO: 7, wherein the variant is the translation of a single nucleotide polymorphism;
(8) a fusion protein comprising a polypeptide according to (1) or (2) or (3);
(9) a fusion protein comprising a polypeptide according to (1) or (2) or (3), wherein said fusion protein comprises one or more amino acid sequence selected from: membrane-bound protein, immunoglobulin constant region, multimerization domains, extracellular proteins, signal peptide-containing proteins, export signal-containing proteins; or
(10) an antagonist of a polypeptide according to (1) or (2) or (3), wherein said antagonist comprises an amino acid sequence containing the non-conservative substitution and/or the deletion of one or more residues in the corresponding polypeptide;
(b) an isolated nucleic acid comprising SEQ ID NO: 1 or SEQ ID NO: 3 or SEQ ID NO: 6, or the complement of said sequence;
(c) a purified nucleic acid which hybridizes under high stringency conditions or exhibits at least about 85% identity over a stretch of at least about 30 nucleotides with a nucleic acid comprising SEQ ID NO: 1 or SEQ ID NO: 3 or SEQ ID NO: 6, or a complement of said sequence;
(d) a vector comprising a nucleic acid as set forth in (a) (or (b) or (c);
(e) a vector comprising a nucleic acid as set forth in (a) or (b) or (c) operatively linked to expression control sequences; and
(f) determining the effect of the compound on the animal or on the cell.

50. The method according to claim 43, wherein said method for identifying a candidate compound as an antagonist/inhibitor or agonist/activator of a polypeptide having IL-8-like/TMEM9 activity comprises:
(a) contacting said polypeptide, said compound, and a mammalian cell or a mammalian cell membrane capable of binding the polypeptide; and
(b) measuring whether the molecule blocks or enhances the interaction of the polypeptide, or the response that results from such interaction, with the mammalian cell or the mammalian cell membrane.

51. The method according to claim 43, wherein said method for determining the activity and/or the presence of a polypeptide having IL-8-like/TMEM9 activity in a sample, the method comprising:
(a) providing a protein-containing sample;
(b) contacting said sample with a ligand; and
(c) determining the presence or said ligand bound to said polypeptide.

52. The method according to claim 43, wherein said method for determining the presence or the amount of a transcript or of a nucleic acid encoding a polypeptide having IL-8-like/TMEM9 activity in a sample, the method comprising:
(a) providing a nucleic acids-containing sample;
(b) contacting said sample with a nucleic acid; and
(c) determining the hybridization of said nucleic acid with a nucleic acid into the sample.
